(19) United States
(12) Patent Application Publication
(10) Pub. No.: US 2024/0294595 A1
(43) Pub. Date: Sep. 5, 2024

(54) GLYCOPEPTIDES AND USES THEREOF

(71) Applicant: Arizona Board of Regents on Behalf of the University of Arizona, Tucson, AZ (US)

(72) Inventors: Robin L. Polt, Tucson, AZ (US); Torsten A E Falk, Tucson, AZ (US); Michael L. Heien, Tucson, AZ (US); John Streicher, Tucson, AZ (US)

(21) Appl. No.: 18/530,028

(22) Filed: Dec. 5, 2023

Related U.S. Application Data
(63) Continuation-in-part of application No. 16/181,129, filed on Nov. 5, 2018, now abandoned.
Application No. 16/181,129 is a continuation-in-part of application No. 15/044,924, filed on Feb. 16, 2016, now Pat. No. 10,117,907.
Application No. 15/044,924 is a continuation-in-part of application No. PCT/US2014/051143, filed on Aug. 14, 2014.
(60) Provisional application No. 61/865,958, filed on Aug. 14, 2013.

(51) Int. Cl. C07K 14/575; A61K 38/00

(52) U.S. Cl. CPC C07K 14/57563; A61K 38/00

(57) ABSTRACT

Glycopeptides comprising a peptide that is covalently linked to a saccharide. The peptide portion of the glycopeptides of the invention has from about 20 to about 40 amino acid residues and at least 75% sequence identity to SEQ ID NO:5. The saccharide moiety portion of the glycopeptides of the present invention comprises from 1 to about 8 carbohydrates. The present invention also relates to using the glycopeptides of the invention in treating various neurodegenerative diseases.

### CROSS-REFERENCE TO RELATED APPLICATIONS

This application is a continuation-in-part (CIP) application of U.S. patent application Ser. No. 16/181,129 filed Nov. 5, 2018, which is a continuation-in-part (CIP) application of U.S. patent application Ser. No. 15/044,924, filed Feb. 16, 2016, which is a CIP application of the US National Phase of PCT Application No. PCT/US2014/051143 filed Aug. 14, 2014, which claims the priority benefit of U.S. Provisional Application Ser. No. 61/865,958, filed Aug. 14, 2013, all of which are incorporated herein by reference in their entirety.

### STATEMENT REGARDING FEDERALLY FUNDED RESEARCH

This invention was made with Government support under Grant No. CHE-9526909 awarded by the National Science Foundation, Grant No. R01-NS052727 and Grant No. R01-NS091238, both awarded by the Department of Health and Human Services, National Institutes of Health, National Institute of Neurological Disorders and Stroke. The Government has certain rights in the invention.

### SEQUENCE LISTING

The instant application contains a Sequence Listing which has been submitted via Patent Center and is hereby incorporated by reference in its entirety. Said .xml copy, created on Mar. 28, 2024 is named UOAZ_P2061 USC3_00597102, and is 226,236 bytes in size.

### FIELD OF THE INVENTION

The present invention relates to glycopeptides comprising a peptide that is covalently linked to a saccharide. In particular, the peptide portion of the glycopeptides of the invention has from about 20 to about 40 amino acid residues and at least 75% sequence identity to SEQ ID NO:1, 2, or 3. The saccharide moiety portion of the glycopeptides of the present invention comprises from 1 to about 8 carbohydrates. The present invention also relates to using the glycopeptides of the invention in treating various neurodegenerative diseases. In particular, glycopeptides of the invention are useful in treating amyotrophic lateral sclerosis (ALS), Parkinson's Disease (PD), Alzheimer's Disease (AD), Huntington's Disease (HD), migraine attacks, traumatic brain injury (TBI) and stroke, as well as certain forms of dementia and eye diseases related to both the cornea and the retina.

### BACKGROUND OF THE INVENTION

Endogenous opioid peptides, lumped together under the generic term endorphins, have been the subject of intense study since their discovery. Neuropeptides have the potential for extremely selective pharmacological intervention with fewer side effects. If these naturally occurring opioid peptides and their derivatives could be rendered permeable to the blood-brain barrier (BBB), then a new vista of psychopharmacology would be opened to exploration and exploitation. After three decades of research, many potent and selective opioid agonists have been developed, and stability problems have been largely overcome. The remaining problem that prevents the use of opioid peptides as drugs is poor bioavailability, which is primarily due to poor penetration of the BBB. The BBB is composed of endothelial cells in the cerebrovascular capillary beds. The BBB acts as a lipophilic barrier to undesired chemical substances, and admits vital nutrients for proper function of the CNS. The flow is bi-directional, allowing for export of materials from the CNS (efflux transport) and the import of materials from the blood (influx transport). The BBB represents not only a physical obstacle, but a metabolic one as well, possessing both oxidative enzymes and peptidases such as aminopeptidase, arylamidase and enkephalinase. Thus, metabolically unstable substances (e.g. peptides) are generally degraded before they reach the CNS. It should also be noted that entry to the CNS does not guarantee that a drug will accumulate in useful concentrations, as many peptides are rapidly exported back to the bloodstream. Several strategies have been reported to overcome the BBB penetration problem, including substitution of unnatural amino acids, the use of conformational constraints, and the addition of lipophilic side chains or other transport vectors.

Glycosylation has proven to be a successful methodology to improve both the stability and bioavailability of short peptide “messages” by incorporation of the peptide pharmacophore into a glycopeptide. Previous BBB penetration studies with opioid glycopeptide agonists based on enkephalins have shown up to 15-fold increases in the rate of brain delivery of these compounds compared with the unglycosylated parent peptides. Recent studies with glycopeptides in artificial membrane systems indicate that amphipathicity of the glycopeptides is an important factor in BBB penetration. In addition, there is evidence that suggests that the type of glycosylation can alter tissue distribution patterns, BBB penetration and peptide/receptor interactions.

The endogenous neuropeptide ß-endorphin is a 31 residue naturally occurring opioid peptide agonist that binds to μ and δ receptors. Its N-terminal 5 residues are identical to the Met-Enkephalin sequence, and may be considered to be the pharmacophore or “opioid message.” It was shown some time ago that the C-terminal region of ß-endorphin has an amphipathic α-helical structure that plays a role in the receptor binding and opioid agonism and may induce resistance to proteolysis. Studies have shown the N-terminal sequence is the essential “message,” and the C-terminal helical region is the “address” that limits delivery of the message to a subset of otherwise available opioid receptors. Some have proposed that ß-endorphin consists of the Met-enkephalin peptide sequence at the N-terminus, a hydrophilic linker region from residues 6 through 12, and an amphiphilic helical region between the helix breaker residues Pro(13)(SEQ ID NO:58) and Gly(30) (SEQ ID NO:59). This was later proven by synthesizing a number of ß-endorphin mimics with artificial C-terminal helical regions with amphipathic character. These de novo amphipathic helices were not homologous with the ß-endorphin C-terminal region, and they were shown to be largely α-helical by circular dichroism (CD) measurements. These hybrid structures showed good opioid agonism in vitro when compared to ß-endorphin. These studies strongly suggested that the overall amphipathicity of the C-terminal helix plays a key role in the selectivity of these compounds, rather than the identity of specific amino acid residues present in the C-terminal.

Dynorphin A (1-17) is also an endogenous opioid peptide, but it binds preferentially to the κ opioid receptor and has an N-terminal message segment identical to Leu-Enkephalin. It has been suggested that an address sequence in the C-terminal region imparts selectivity for κ receptors. Dynorphin A displayed an extended and/or random coil structure when subjected to structural analysis by various spectroscopic measurements. A 2D (1) H-NMR study in DPC micelle showed that Dynorphin A(1-17) contains a less ordered N-terminal segment, a well defined α-helix segment spanning between Phe(4) (SEQ ID NO:60) and Pro(10) (SEQ ID NO:61) or Lys(11) (SEQ ID NO:62), and a ß-turn from Trp(14) (SEQ ID NO:63) to Gln(17) (SEQ ID NO:64). Based on NMR results, some believe that both the α-helix and the C-terminal ß-turn are due to dynorphin-micelle interactions, and may be important structural features of the full-length peptide when bound to the cell membrane in vivo, and this membrane bound conformation is essential for biological activity at the GPCR target.

Studies by others also support the notion that a helical structure in the message segment of Dynorphin A(1-17) is significant. The biological importance of helical C-terminal address segments in larger opioid peptides has been further supported by the recent work by Kyle and co-workers. They successfully synthesized several potent nociceptin (NC) peptide analogs exploiting the α-helix-promoting residues α-aminoisobutyric acid (Aib) and N-methyl alanine (MeAla) at the C-terminus of NC.

Nociceptin is the endogenous ligand for the recently identified opioid receptor-like 1 receptor (ORL-1). Thus, it seems logical to approach the design of opioid agonist ß-endorphin or dynorphin peptide analogs by combining C-terminal amphipathic helical address segments that can also promote BBB, for penetration by virtue of glycosylation.

It is an object of the present invention to provide glycopeptides that can readily penetrate the blood-brain-barriers (BBB) for treatment of neurodegenerative diseases such as amyotrophic lateral sclerosis (ALS), Parkinson's Disease (PD), Alzheimer's Disease (AD), Huntington's Disease (HD), migraine attacks, traumatic brain injury (TBI) and stroke, as well as certain forms of dementia and eye diseases.

### SUMMARY OF THE INVENTION

One aspect of the invention provides a glycopeptide comprising a peptide that is covalently linked to a saccharide, wherein said peptide has from about 20 to about 40 amino acid residues and at least 75% sequence identity to pleiotropic peptide pituitary adenylate cyclase-activating polypeptide (PACAP) of SEQ ID NOs: 1 or 2, or vasoactive intestinal peptide (VIP) of SEQ ID NO:3, and wherein said saccharide comprises from 1 to about 8 carbohydrates.

In one particular embodiment, the glycopeptide is selected from the group consisting of PACAP₁₋₂₇, PACAP₁₋₃₈, VIP, [AC-His¹]PACAP-27, [Ala²]PACAP-27, [Gly²⁰]PACAP-27, and Ac-[Phe(pI)⁶, Nle¹⁷]-PACAP₁₋₂₇. Within this embodiment, the glycopeptide is [Ala²]PACAP-27 is a D-isomer alanine, i.e., [D-Ala²]PACAP-27.

Still in another embodiment, at least one of the amino acid residues of SEQ ID NOs:1, 2 or 3 is substituted with a substitution amino acid residue having a side-chain functional group that is glycosylated. Typically, the substitution amino acid residue comprises serine, threonine, hydroxyproline or a similar ethanolamine linker.

In some embodiments, the glycopeptide has from about 25 to about 40, typically from about 25 to about 35, often about 25 to about 30, and more often 27 to 29 amino acid residues.

Yet in other embodiments, the glycopeptide has at least 80%, typically 85%, and often 90% sequence identity to PAPAC₁₋₂₇ (SEQ ID NO:2).

In one embodiment, the present disclosure provides a glycopeptide, which comprises a polypeptide and one or more saccharide molecules covalently linked to the polypeptide, wherein the glycopeptide is capable of crossing human blood brain barrier (BBB), and the polypeptide has a sequence of:

(SEQ ID NO: 5)
HSDX⁴X⁵FX⁷DSYSRYRKQX¹⁷AVKKYLAAX²⁶X²⁷x²⁸

wherein
   X⁴ is glycine; alanine; D-alanine; sarcosine; β-alanine; or diaminovaleric acid;
   X⁵ is isoleucine; valine; leucine; L-tert-Leucine; L-nor-Valine; L-Nor-Leucine; glycine;
sarcosine; D- or L-alanine; or D- or L-N-methylalanine;
   X⁷ is threonine or alanine;
   X¹⁷ is L-Nor-Leucine, L-Leucine; Valine; Nor-Valine; Alanine; or one of the other aliphatic amino acids, or Glycine;
   X²⁶ is valine or leucine;
   X²⁷ is leucine or serine; and
   X²⁸ is absent or serine,
and wherein at least one amino acid residue of the polypeptide is glycosylated.

In another embodiment, a glycopeptide is disclosed which comprises a polypeptide with the same amino acid sequence as SEQ ID NO: 5 except that X¹⁷ may be any amino acids other than Methionine. In one aspect, X¹⁷ is Nor-Leucine, Leucine; Valine; Nor-Valine; Alanine; or Glycine. In another aspect, the X¹⁷ is L-Nor-Leucine, or L-Leucine.

In another embodiment, the at least two of the amino acids are glycosylated. In one aspect, the saccharide molecules covalently linked to the polypeptide contains 1-20, or 3-10, or 5-8 monosaccharide units. In one aspect, the saccharide molecules covalently linked to the polypeptide contains at least 2, or at least 3 monosaccharide units.

In another embodiment, serine at 2-position of the glycopeptide is (D)-isomer.

In another embodiment, the X²⁸ of the glycopeptide is Serine. In one aspect, the Serine-28 is glycosylated with glucose, galactose, melibiose, xylose, lactose, trehalose, or altose.

In another embodiment, the polypeptide has a amino acid sequence that is at least 75%, at least 90%, at least 95%, or 100% identical to the sequence selected from the group consisting of:
HSDGIFTDSYSRYRKQLAVKKYLAAVL-Ser-CONH₂ (SEQ ID NO: 6, referred to as 2LS80 in this disclosure), and
HSDGIFTDSYSRYRKQLAVKKYLAAVL-Ser-CONH₂ (SEQ ID NO: 7, referred to as 2LS98 in this disclosure) wherein Ser-28 and at least one amino acid other than Ser-28 are glycosylated.

In another embodiment, the glycopeptide comprising the polypeptide having SEQ ID NO: 6 or SEQ ID NO: 7 has Ser-28 that is glycosylated with melibiose, or lactose.

In yet other embodiments, the C-terminus end of the peptide is glycosylated.

Still yet in other embodiments, the glycopeptide is a pituitary adenylate cyclase-activating polypeptide type I receptor (PAC₁) agonist. In other embodiments, the glycopeptide is a VPAC₁ agonist. Yet in other embodiments, the glycopeptide is a selective PAC₁ and VPAC₁ agonist.

Yet in other embodiments, the PAC1 binding affinity (Ki) of the glycopeptide is less than about 10 nM.

In other embodiments, the VPAC1 binding affinity (Ki) of the glycopeptide is less than about 10 nM.

Still in other embodiments, the PAC1 agonist activity (Ki) of the glycopeptide is less than about 10 nM.

Yet in other embodiments, the VPAC1 agonist activity (Ki) of said glycopeptide is less than about 10 nM.

Typically, the saccharide comprises from 1 to 5, often 1 to 3 carbohydrates. In one particular embodiment, the saccharide is a monosaccharide, a disaccharide.

Still yet in other embodiments, the peptide comprises a plurality of glycosylated amino acid residues, i.e., different amino acids are glycosylated where each saccharide linked to the amino acid residue is independently selected. In some embodiments, 1 to 5, typically 1 to 3, often 1 or 2 different amino acid residues are glycosylated.

In some embodiments, the saccharide is selected from the group consisting of glucose, maltose, lactose, melibiose, maltotriose, sucrose, trehalose, altose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, polysaccharides related to the Thompsen-Friedrich antigens (Tn), and other monosaccharide or a disaccharide described herein.

In one embodiment, the peptide is a peptide of SEQ ID NO:2 with 0-5, typically 0-3, often 1-3, more often 1 or 2 and most often 1 additional amino acid residue(s).

In another embodiment, the disclosed glycopeptide is capable of penetrating blood-brain barrier (BBB) and reaching a CSF concentration of at least 50 nM, or 100 nM, or 400 nM 60 minutes after being injected into a subject intravenously or subcutaneously at a concentration of 15 mg glycopeptide per kg body weight of the subject.

Another aspect of the invention provides a method for treating a neurodegenerative disease in a subject. The method comprises administering to the subject in need of such a treatment a therapeutically effective amount of a glycopeptide of the present invention. The glycopeptide of the invention has a higher blood-brain barrier penetration compared to the same peptide in the absence of a saccharide.

In other embodiments, the glycopeptide of the invention has a higher stability compared to the same peptide in the absence of the saccharide.

In other embodiments, the glycopeptide of the invention has a higher penetration of the BBB as compared to the same peptide in the absence of the saccharide.

Still in other embodiments, neurodegenerative disease is selected from the group consisting of amyotrophic lateral sclerosis, Parkinson's Disease, migraine attacks, traumatic brain injury, stroke, and dementia.

### BRIEF DESCRIPTION OF THE DRAWINGS

FIG. 1 is a graph showing stability of PACAP₁₋₂₇, PACAP_{1-27-S-G} and PACAP-27-S-L;

FIG. 2 is a bar graph showing half-life of PACAP₁₋₂₇ and its corresponding serine glucosides in mouse serum;

FIG. 3 shows PAC₁-CHO calcium flux of PACAP₁₋₂₇ and various truncated derivatives;

FIGS. 4A-4C (counter clock-wise from top left, respectively) are graphs showing PACAP₁₋₂₇ and glycopeptides of the invention stimulation at various concentrations; and

FIGS. 5A-5D show PC12 cell morphology after vehicle versus PACAP treatment at various solutions.

FIG. 6 shows synthetic scheme for preparation of PACAP-derived glycopeptide.

FIGS. 7A-7B shows results of in vivo stability and BBB transport. (FIG. 7A) In vivo CSF concentrations of d82L98-OH and d82LS98Lact following 15 mg/kg i.v. injection in rats (n=5). Glycopeptide concentrations were quantified by HPLC-MS. (FIG. 7B) Area under the curve (AUC) of d82L98-OH and d82LS98Lact following 15 mg/kg i.v. injection in rats. AUCs are normalized to d82L98-OH. d82L98-OH and d82LS98Lact are deuterated mass-shifted analogues of 2L98-OH and 2LS98Lact. Dosage of 15 mg/kg are used to overcome the high detection of limit in CSF. Glycosylated analogues exhibited enhanced BBB transportation with a disaccharide moiety. Concentrations and AUCs are reported by mean±SEM. Asterisks demote statistical significance using repeated measures Mann-Whitney test, p<0.01.

FIGS. 8A-8O shows results of protective and anti-inflammatory effects of 2LS80Mel in a mouse model of TBI. Mice were subjected to diffuse TBI or control sham surgery and treated with either 2LS80Mel or sterile saline. The in vivo efficacy of 2LS80Mel was then evaluated by assessing its effects on the sleep-wake behavior (FIG. 8A-FIG. 8C), neurological and motor skill deficits (FIG. 8D-FIG. 8F), monocyte populations (FIG. 8G-FIG. 8I), concentrations of inflammatory cytokines (FIG. 8J-FIG. 8L), and microglial morphology (FIG. 8M-FIG. 8O). Overall, 2LS80Mel attenuated behavioral, neurological, and motor skill deficits. Furthermore, 2LS80Mel prevented increases in peripheral monocyte populations. There were no significant differences in inflammatory cytokine concentrations or microglial ramification between the 2LS80Mel-treated, untreated, and sham animals.

FIGS. 9A-9N shows results of neuroprotection of the PACAP glycopeptide 2LS98Lac in rat Parkinson's disease models. (FIG. 9A) Scheme for study 1, using a mild PD lesion. (FIG. 9B) Scheme for study 2, using a moderate PD lesion. (FIG. 9C) Systemic injection (i.p.) of 2LS98Lac reduces 6-OHDA-induced lesion damage in the mild 6-OHDA hemi-parkinsonian rat model. Amphetamine-induced rotations at 2- and 4-weeks post-lesion are plotted (mean rotations±SEM). (FIG. 9D) The mean±SEM cumulative amphetamine-induced rotations are plotted showing that treatment with the PACAP glycopeptide reduced the number of rotations indicative of a protective effect *p<0.05. (FIG. 9E) Unbiased stereology of TH-positive dopaminergic neurons in the substantia nigra (SNc) in study 1 reveal a significant 6-OHDA-induced loss of TH-positive neurons on the lesioned side in the vehicle control group (V), but not the 2LS98Lac-treated group (P). *p<0.05. (FIG. 9F, FIG. 9G) Systemic injection (i.p.) of 2LS98Lac does not reduce 6-OHDA-induced lesion damage in the moderate 6-OHDA hemi-parkinsonian rat model. Amphetamine-induced rotations at 2 (FIG. 9F) and 4 (FIG. 9G) weeks post-lesion are plotted (mean rotations±SEM). (FIG. 9H) Unbiased stereology of TH-positive neurons in the SNc in study 2 reveal a significant 6-OHDA-induced loss of TH-positive neurons on the lesioned side for both groups (V and P), but no group difference. ***p<0.001. (FIG. 9I) 2LS98lac-treatment did not change mean (±SEM) striatal dopaminergic content (DA) analyzed with HPLC-EC. Mean data (±SEM) are plotted as % control. (FIG. 9J) 2LS98lac-treatment did not change striatal TH expression, quantified with semi-quantitative western analysis with beta-actin (βA) as internal standard. Mean data (±SEM) are plotted as % control. (FIG. 9K-FIG. 9N) 2LS98lac rescues 6-OHDA induced morphological changes to microglia (mean data±SEM). Specifically, we show % area IBA1/cell (FIG. 9K), process length/cell (FIG. 9L), number of branches/cell (FIG. 9M) and endpoints/cell (FIG. 9N). *p<0.05; **p<0.01.

### DETAILED DESCRIPTION OF THE INVENTION

As used in this specification and the appended claims, the singular forms “a,” “an” and “the” include plural references unless the content clearly dictates otherwise.

The terms “saccharide” and “carbohydrate” are used interchangeably herein and refer to aldoses and ketoses consisting of carbon (C), hydrogen (H) and oxygen (O) atoms, typically, but not necessarily, with a hydrogen-oxygen atom ratio of 2:1. The term also includes mono-deoxy-carbohydrates, such as deoxyribose, etc. where one hydroxy group is removed from the empirical formula Cₘ(H₂O)ₙ formula, where m is typically 6 and n can be 5 or 6.

The terms “sugar” refers to a mono- and/or disaccharide.

The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₆H₁₂O₆ or a derivative thereof. Exemplary aldoses of the invention include, but are not limited to, allose, altrose, glucose, mannose, gulose, idose, galactose, talose, ribose, arabinose, xylose, lyxose, and derivatives thereof. Exemplary ketoses of the invention include, but are not limited to, psicose, fructose, sorbose, tagatose, ribulose, xylulose, and derivatives thereof. As used herein the term “derivative” refers to a derivative of a monosaccharide in which one or more of the hydroxyl groups is replaced with hydrogen (e.g., 2-deoxy glucose, 5-deoxyglucose, etc.), an amine (e.g., amino sugars) or is replaced with a halogen, such as chloro, fluoro or iodo, (e.g., 5-fluoroglucose, 2-fluoroglucose, 5-chrologlucose, 2-chloroglucose, etc.). Monosaccharide can be an (L)-isomer or a (D)-isomer.

The term “disaccharide” refers to a carbohydrate composed of two monosaccharides. It is formed when two monosaccharides are covalently linked to form a dimer. The linkage can be a (1→4) bond, a (1→6) bond, a (1→2) bond, etc. between the two monosaccharides. In addition, each of the monosaccharides can be independently an α- or β-anomer. Exemplary disaccharides that can be used in the present invention include, but are not limited to, sucrose, lactose, altose, maltose, trehalose, cellobiose, lactulose, and chitobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Exemplary aldoses that can be used in preparing disaccharides of the invention include, but are not limited to, allose, altrose, glucose, mannose, gulose, idose, galactose, talose, ribose, arabinose, xylose, lyxose, and derivatives thereof. Exemplary ketoses that can be used in preparing disaccharides of the invention include, but are not limited to, psicose, fructose, sorbose, tagatose, ribulose, xylulose, and derivatives thereof. Each monosaccharide can also be independently an (L)-isomer or a (D)-isomer.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

As used herein, the term “treating”, “contacting” or “reacting” when referring to a synthesis or chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The terms “identical,” “identity,” “percent identity,” “percent sequence identity,” and “sequence identity” are used interchangeably herein. In particular, in the context of comparison of two or more peptides, these terms refer to two or more sequences or subsequences that are the same or have a specified percentage of amino acid residues that are the same, when compared and aligned for maximum correspondence, as measured using a sequence comparison algorithm known to one skilled in the art, or by visual inspection. For example, 75% sequence identity of a peptide A compared to peptide B means, 75% of the amino acid sequences in peptide A are the same as that of the amino acid sequences of peptide B. The term also includes insertion/addition or deletion of amino acids compared to a reference peptide. Thus, 75% sequence identity of peptide A compared to peptide B can also mean that peptide A has 25% more or 25% less (i.e., ±25%) amount of amino acid residues. In particular, if peptide B has 27 amino acids, then 75% sequence identify of peptide A means peptide A can have from 21 to about 33 amino acid residues. In some embodiments, these terms are used to denote sequences which when aligned have similar (identical or conservatively replaced) amino acids in like positions or regions, where identical or conservatively replaced amino acids are those which do not alter the activity or function of the protein as compared to the starting protein. Percent sequence identity may be calculated by determining the number of residues that differ between a peptide encompassed by the present invention and a reference peptide such as SEQ ID NOS: 1, 2 or 3), taking that number and dividing it by the number of amino acids in the reference peptide (e.g., 27 or 27 amino acids), multiplying the result by 100, and subtracting that resulting number from 100. For example, a sequence having 35 amino acids with four amino acids that are different from VIP would have a percent (%) sequence identity of 89% (e.g. 100−((4/35)×100)). For a peptide having a sequence that is longer than the number of amino acids in a reference peptide, the number of residues that differ from the reference peptide will include the additional (or difference in) amino acids over (or under) 35 for purposes of the aforementioned calculation. For example, a sequence having 37 amino acids, with four amino acids different from the 35 amino acids in the reference peptide sequence and with two additional amino acids at the carboxy terminus which are not present in the reference peptide sequence, would have a total of six amino acids that differ from the reference peptide. Thus, this sequence would have a percent (%) sequence identity of 83% (e.g. 100−((6/35)×100)). The degree of sequence identity may be determined using methods well known in the art (see, for example, Wilbur, W. J. et al., Proc. Natl. Acad. Science USA, 1983, 80, 726-730 and Myers E. et al., Comput. Appl. Biosci., 1988, 4, 11-17. One program which may be used in determining the degree of similarity is the MegAlign Lipman-Pearson one pair method (using default parameters) which can be obtained from DNAstar Inc, 1128, Selfpark Street, Madison, Wisconsin, 53715, USA as part of the Lasergene system. Another program, which may be used, is Clustal W. This is a multiple sequence alignment package developed by Thompson et al. (Nucleic Acids Research, 1994, 22(22), 4673-4680) for DNA or protein sequences. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “about” as used herein is not intended to limit the scope of the invention but instead encompass the specified material, parameter or step as well as those that do not materially affect the basic and novel characteristics of the invention. The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of ±20%, typically ±10%, often ±5% and more often ±1% of a given numeric value.

The present invention provides glycopeptides that are stable and can cross the blood-brain barrier. Glycopeptides are peptides that contain carbohydrate moieties (glycans or saccharides) covalently attached to the side chains of the amino acid residues that constitute the peptide. In particular, glycopeptides of the invention include a peptide that is covalently linked to a saccharide. The peptide portion of the glycopeptide of the invention has from about 20 to about 40 amino acid residues and at least 75% sequence identity to pleiotropic peptide pituitary adenylate cyclase-activating polypeptide (PACAP) of SEQ ID NO:1 or vasoactive intestinal peptide (VIP) of SEQ ID NO:3. The saccharide portion of the glycopeptide of the invention ranges from 1 to about 8 carbohydrates.

PACAP is a neuropeptide consisting of 38 amino acids. Two forms of the PACAP are known: PACAP-38 (i.e., PACAP₁₋₃₈) (SEQ ID NO:1) and PACAP-27 (i.e., PACAP₁₋₂₇) (SEQ ID NO:2):

(SEQ ID NO: 1)
1 10 20 30 38
HSDGIFTDSY SRYRKQMAVK KYLAAVLGKR YKQRVKNK
(SEQ ID NO: 2)
1 10 20 27
HSDGIFTDSY SRYRKQMAVK KYLAAVL

As the name indicates, PACAP-38 is a full-length PACAP having 38 amino acid residue (i.e., SEQ ID NO:1) and PACAP-27 (i.e., amino acid residues one to twenty-seven of SEQ ID NO:1) is a shorter version of PACAP. Interestingly, PACAP-27 has a 68% homology to VIP (SEQ ID NO:3), which has 28 amino acid residues:

(SEQ ID NO: 3)
1 10 20 28
HSDAVFTDNY TRLRKQMAVK YYLNSILN

PACAP was first isolated from bovine hypothalamus, and is known to regulate the development, maintenance, function, and plasticity of the nervous system, providing neuroprotective and neurotrophic support. PACAP has been shown to activate 3 closely-related G protein coupled receptors: (i) PAC₁, which has much higher affinity for PACAP, (ii) VPAC₁, and (iii) VPAC₂ which bind both PACAP and VIP. They are expressed on neurons, microglia, and also by many other cell types. Without being bound by any theory, it is believed constitutive expression of PACAP and its receptor PAC₁ confers neuroprotection to central visceromotor neurons via the PAC₁ receptor. PACAP also promotes cytodestructive functions of microglia (M1 amoeboid→M2 hypertrophic phenotype), thought to drive ALS disease progression via the VPAC₁ receptor. Thus, the ideal drugs for neuroprotection would be PAC₁ agonists at motor neurons to promote neuroprotection in case of ALS, or dopaminergic neurons in case of PD, or hippocampal neurons in case of AD, and in each case VPAC₁ antagonists at microglia to reduce inflammation by maintaining the M1 (‘alternatively activated’/resolving anti-inflammatory cells) phenotype vs. the M2 (the classical, proinflammatory macrophages) microglia phenotype or Tau-opathies.

The present invention is based at least in part in providing treatments for ALS, PD, AD, and/or HD. In one aspect of the invention, a glycopeptide that can be used to treat a neurodegenerative disease. In particular, the glycopeptide of the invention includes a peptide that is covalently linked to a saccharide. The peptide portion of the glycopeptides of the present invention has from about 20 to about 40 amino acid residues and at least 75% sequence identity to pleiotropic peptide pituitary adenylate cyclase-activating polypeptide (PACAP) of SEQ ID NOs:1 or 2; or vasoactive intestinal peptide (VIP) of SEQ ID NO:3. The saccharide portion of the glycopeptides of the invention include from 1 to about 8 carbohydrates.

In one embodiment, the glycopeptide is selected from the group consisting of PACAP₁₋₂₇, PACAP₁₋₃₈, VIP, [AC-His¹]PACAP-27, [Ala²]PACAP-27, [Gly²⁰]PACAP-27, and Ac-[Phe(pI)⁶, Nle¹⁷]-PACAP₁₋₂₇. It should be appreciated, the terms [Ac-His¹]PACAP-27, [Ala²]PACAP-27, [Gly²⁰]PACAP-27, and Ac-[Phe(pI)⁶, Nle¹⁷]-PACAP₁₋₂₇ have the conventional meaning well known to one skilled in the art. For example, [Ac-His¹]PACAP-27 means the histidine residue 1 in PACAP-27 is acetylated; [Ala²]PACAP-27 means the second amino acid residue of PACAP-27, which is serine, is replaced by alanine; similarly [Gly²⁰]PACAP-27 means the 20^{th} amino acid residue of PACAP-27, namely, lysine, is replaced with glycine; and Ac-[Phe(pI)⁶, Nle¹⁷]-PACAP₁₋₂₇ means amino acid residues 6 and 17 of PACAP-27, namely, Phenylalanine and methionine, respectfully, are replaced with para-iodo phenylalanine and norleucine (“Nle”), respectively.

In some embodiments, the glycopeptide of the invention is a retro modified peptide. The term “retro modified” refers to a peptide which is made up of L-amino acids in which the amino acid residues are assembled in opposite direction to the native peptide with respect the which it is retro modified. In other embodiments, the glycopeptide of the invention is an inverso modified peptide. The term “inverso modified” refers to a peptide which is made up of D-amino acids in which the amino acid residues are assembled in the same direction as the native peptide with respect to which it is inverso modified. Still in other embodiments, the glycopeptide of the invention is a retro-inverso modified peptide. The term “retro-inverso modified” refers to a peptide which is made up of D-amino acids in which the amino acid residues are assembled in the opposite direction to the native peptide with respect to which it is retro-inverso modified. Thus, for example, if a normal (i.e., native) Link-N peptide (L-amino acids, N—C direction) is HSDGIFTDSY(SEQ ID NO: 8), then Retro-inverso Link-N peptide (D-amino acids, C—N direction) is: HSDGIFTDSY(SEQ ID NO: 8). Retro peptide (L-amino acids, C—N direction) is: HSDGIFTDSY(SEQ ID NO: 8). And inverso peptide (D-amino acids, N—C direction) is: HSDGIFTDSY(SEQ ID NO: 8). These modifications allow inter alia greater stability of peptides from enzymatic and/or hydrolytic cleavage in vivo. In some embodiments, one or more, typically one to ten, often one to five, and more often one to three amino acid residues of the glycopeptide of the invention can be a D-isomer. In one particular embodiment, a single amino acid reside is replaced with a D-isomer.

In one particular embodiment, glycopeptide [Ala²]PACAP-27 includes a D-isomer alanine, i.e., [D-Ala²]PACAP-27.

Still in another embodiment, at least one of the amino acid residues of SEQ ID NO:1, 2 or 3 is substituted with a substitution amino acid residue having a side-chain functional group that is glycosylated. Typically, the substitution amino acid residue comprises serine, threonine, hydroxyproline or a similar ethanolamine linker. As used herein, the term “substitution amino acid” refers an amino acid residue that is used to replace another amino acid in the same position. In contrast, the term “additional amino acid residue” refers to an amino acid residue that is added (i) in between two amino acid residues, (ii) at the C-terminus end of, or (iii)N-terminus end of SEQ ID NOs:1, 2 or 3. In a similar manner, the term “deletion” or “deleted” amino acid residue refers to an amino acid residue that is deleted or removed (i) from between two amino acid residues, (ii) at the C-terminus end of, or (iii)N-terminus end of SEQ ID NOs:1, 2 or 3.

In some embodiments, the glycopeptide has from about 25 to about 40, typically from about 25 to about 35, often about 25 to about 30, and more often 27 to 29 amino acid residues. In one particular embodiment, the glycopeptide has from about 25 to 30 amino acid residue and at least 75%, typically at least 80%, often at least 85%, and more often at least 90% sequence identity to SEQ ID NO:2.

Yet in other embodiments, the glycopeptide has at least 80%, typically 85%, and often 90% sequence identity to PAPAC₁₋₂₇ (SEQ ID NO:2).

Still yet in another embodiment, the glycopeptide of the invention has at least 75%, typically at least 80%, often at least 85%, and more often at least 90% sequence identity to SEQ ID NO:2 and includes at least one, typically one to five, often one to three, and more often one additional amino acid residue. In one particular embodiment, glycopeptides of the invention have a 28 amino acid residue and at least 75%, typically at least 80%, often at least 85%, and more often at least 90% sequence identity to SEQ ID NO:2.

Still in other embodiments, the glycopeptide of the present invention has at least one, typically, one to ten, often one to five, and more often one to five additional amino acid residues of SEQ ID NOs:1, 2, or 3.

In yet other embodiments, the C-terminus end of the peptide is glycosylated.

Still yet in other embodiments, the glycopeptide is a pituitary adenylate cyclase-activating polypeptide type I receptor (PAC₁) agonist. In other embodiments, the glycopeptide is a VPAC₁ agonist. Yet in other embodiments, the glycopeptide is a selective PAC₁ and VPAC₁ agonist. Still in other embodiments, the glycopeptides of the invention are PAC₁ agonist and VPAC₁ antagonist.

Yet in other embodiments, the PAC1 binding affinity (Ki) of the glycopeptide is less than about 50 nM, typically less than about 25 nM, and often less than about 10 nM.

In other embodiments, the VPAC1 binding affinity (Ki) of the glycopeptide is less than about 50 nM, typically less than about 25 nM, and often less than about 10 nM.

Still in other embodiments, the PAC1 agonist activity (Ki) of the glycopeptide is less than about 50 nM, typically less than about 25 nM, and often less than about 10 nM.

Yet in other embodiments, the VPAC1 agonist activity (Ki) of said glycopeptide is less than about 50 nM, typically less than about 25 nM, and often less than about 10 nM.

Agonistic and/or antagonistic activity of glycopeptides of the invention can be readily determined by one skilled in the art having read the present disclosure. For example, methods described by Doan et al., in Biochemical Pharmacology, 2011, 81, pp. 552-561, as well as in the Examples section below.

Typically, the saccharide comprises from 1 to 5, often 1 to 3 carbohydrates. In one particular embodiment, the saccharide is a monosaccharide or a disaccharide.

Still yet in other embodiments, the peptide comprises a plurality of glycosylated amino acid residues, i.e., different amino acid residues are glycosylated where each saccharide linked to the amino acid residue is independently selected from those described herein. In some embodiments, 1 to 5, typically 1 to 3, often 1 or 2 different amino acid residues are glycosylated. Often one amino acid residue is glycosylated.

In some embodiments, the saccharide is selected from the group consisting of with glucose, maltose, lactose, melibiose, maltotriose, sucrose, trehalose, altose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, polysaccharides related to the Thompsen-Friedrich antigens (Tn), and other monosaccharide and a disaccharide described herein.

In one particular embodiment, the glycopeptide of the invention includes a peptide portion having at least 75% sequence identity to SEQ ID NO:2 and with 0-5, typically 0-3, often 1-3, more often 1 or 2 and most often 1 additional amino acid residue(s).

Still in other embodiments, the glycopeptide of the invention has a peptide portion that is 27 or 28 amino acid residues in length and has about six or less, typically about five or less, and often four or less amino acid residue difference compared to SEQ ID NO:2. Yet in other embodiments, the glycopeptide of the invention has a peptide portion having one additional amino acid residue compared to SEQ ID NO:2. In some embodiments, the additional amino acid residue is attached to the C-terminal end of SEQ ID NO:2. Yet in other embodiments, the additional amino acid residue is glycosylated, i.e., covalently linked to a saccharide.

In other embodiments, glycopeptide of the invention has at least 75%, typically at least 80%, often at least 85%, more often at least 90%, and most often at least 95% sequence identity to SEQ ID NO:4:

(SEQ ID NO: 4)
1 10 20 27 28
HSDXXFXDSY SRYRKQXAVK KYLAAXX X

As can be seen, SEQ ID NO:4 is similar to SEQ ID NO:2 with one additional amino acid residue at the c-terminal end. Still in other embodiments, glycopeptide of the invention is SEQ ID NO:4.

Methods for preparing glycopeptides of the invention can be readily apparent to one skilled in the art having read the present disclosure. One method of preparing glycopeptides of the invention is using Fmoc-based solid-phase peptide synthesis. Briefly, in this method involves covalently attaching a glycosyl group to the amino acid sequence by an O-linkage to a side chain in the address segment of the sequence. See, for example, as described by the present inventor in Tetrahedron Asymmetry, 2005, 16, pp. 65-75, and the commonly assigned U.S. Pat. No. 5,767,254, issued to the present inventor, Robin Polt, all of which are incorporated herein by reference in their entirety. An amino acid residue having a desired saccharide can be prepared using the procedure described in the above cited U.S. Pat. No. 5,767,254. The peptide portion was synthesized using a solid state synthesis procedure as described in Tetrahedron Asymmetry, 2005, 16, pp. 66-75. By adding/linking each desired amino acid to the solid phase synthesis and using a desired glycosylated amino acid at an appropriate step of the synthetic process, one can produce a wide range of glycopeptides of the invention such as those exemplified in Table 1.

Yet in another embodiment, one or more serine amino acid residue of SEQ ID NO:2 or 4 is glycosylated. In one particular embodiment, the saccharide moiety within this embodiment is independently selected from the group consisting of glucose, galactose, melibiose, xylose, lactose, trehalose, altose, or other monosaccharide or a disaccharide described herein. Yet in another particular embodiment, one or more amino acid, e.g., serine, of SEQ ID NO:2 or 4 can be independently either L- or D-isomer.

Still in another embodiment, the glycopeptide is selected from those listed in Table 1 with a proviso that at least one amino acid residue is glycosylated. As can be seen, all glycopeptides in Table 1 fall within the scope of SEQ ID NO:4.

TABLE 1
Representative glycopeptides of SEQ ID NO: 4

SEQ ID NO: 4 Representative Amino Acid Sequences
H S D G I F T D S¹ Y S¹ R Y R K Q L A V K K Y L A A V L [symbol] SEQ ID NO: 16
H s D G I F T D S¹ Y S¹ R Y R K Q [symbol] A V K K Y L A A V L S* SEQ ID NO: 17
AcH s D G I F T D S¹ Y S¹ R Y R K Q [symbol] A V K K Y L A A V L S* SEQ ID NO: 18
H s D G I F T D S¹ Y S¹ R Y R K Q [symbol] A V K K Y L A A V S* SEQ ID NO: 19
H s D G I F T D S¹ Y S¹ R Y R K Q [symbol] A V K K Y L A A L S* L SEQ ID NO: 20
H s D G I F A D S Y S R Y R K Q [symbol] A V K K Y L A A V L S* SEQ ID NO: 21
H s D G I F A D S Y S R Y R K Q [symbol] A V K K Y L A A V S* SEQ ID NO: 22
H s D G I F A D S Y S R Y R K Q [symbol] A V K K Y L A A V S* L SEQ ID NO: 23
H s D a I F T D S Y S R Y R K Q [symbol] A V K K Y L A A V L S* SEQ ID NO: 24
H s D Sar IF T D S Y S R Y R K Q [symbol] A V K K Y L A A V L S* SEQ ID NO: 25
H s D βA I F T D S Y S R Y R K Q [symbol] A V K K Y L A A V L S* SEQ ID NO: 26
H s D Dava I F T D S Y S R Y R K Q [symbol] A V K K Y L A A V L S* SEQ ID NO: 27
H s D A I F T D S Y S R Y R K Q [symbol] A V K K Y L A A V L S* SEQ ID NO: 28
H s D B I F T D S Y S R Y R K Q [symbol] A V K K Y L A A V L S* SEQ ID NO: 29
H s D G V F T D S Y S R Y R K Q [symbol] A V K K Y L A A V L S* SEQ ID NO:30
H s D G L F T D S Y S R Y R K Q [symbol] A V K K Y L A A V L S* SEQ ID NO: 31
H s D G A F T D S Y S R Y R K Q [symbol] A V K K Y L A A V L S* SEQ ID NO: 32
H s D G tL F T D S Y S R Y R K Q [symbol] A V K K Y L A A V L S* SEQ ID NO: 33
H s D G Nva F T D S Y S R Y R K Q [symbol] A V K K Y L A A V L S* SEQ ID NO: 34
H s D G [symbol] F T D S Y S R Y R K Q [symbol] A V K K Y L A A V L S* SEQ ID NO: 35
H s D G G F T D S Y S R Y R K Q [symbol] A V K K Y L A A V L S* SEQ ID NO: 36
H s D Sar G F T D S Y S R Y R K Q [symbol] A V K K Y L A A V L S* SEQ ID NO: 37
H s D G Sar F T D S Y S R Y R K Q [symbol] A V K K Y L A A V L S* SEQ ID NO: 38
H s D Sar Sar F T D S Y S R Y R K Q [symbol] A V K K Y L A A V L S* SEQ ID NO: 39
H s D Sar A F T D S Y S R Y R K Q [symbol] A V K K Y L A A V L S* SEQ ID NO: 40
H s D A Sar F T D S Y S R Y R K Q [symbol] A V K K Y L A A V L S* SEQ ID NO: 41
H s D Sar a F T D S Y S R Y R K Q [symbol] A V K K Y L A A V L S* SEQ ID NO: 42
H s D a Sar F T D S Y S R Y R K Q [symbol] A V K K Y L A A V L S* SEQ ID NO: 43
H s D A Å F T D S Y S R Y R K Q [symbol] A V K K Y L A A V L S* SEQ ID NO: 44
H s D a Å F T D S Y S R Y R K Q [symbol] A V K K Y L A A V L S* SEQ ID NO: 45
H s D A Å′ F T D S Y S R Y R K Q [symbol] A V K K Y L A A V L S* SEQ ID NO: 46
H s D a Å′ F T D S Y S R Y R K Q [symbol] A V K K Y L A A V L S* SEQ ID NO: 47
H S D G I F T D S¹ Y S¹ R Y R K Q M A V K K Y L A A V L [symbol] SEQ ID NO: 48
H S D G I F T D S¹ Y S¹ R Y R K Q M A V K K Y L A A V L S¹ SEQ ID NO: 49
H S¹ D G I F T D S¹ Y S¹ R Y R K Q X¹⁷ A V K K Y L A A V L S¹ SEQ ID NO: 50
H s¹ D G I F T D s/S¹ Y S¹ R Y R K Q M A V K K Y L A A V L S¹ SEQ ID NO: 51
H S¹ D G I F T D S¹ Y S¹ R Y R K Q M A V K K Y L A A V L s/S¹ SEQ ID NO: 52
H S D G I F T D S¹ Y S¹ R Y R K Q M A V K K Y L A A V [symbol] SEQ ID NO: 53
H S D G I F T D S¹ Y S¹ R Y R K Q M A V K K Y L A A V S¹ SEQ ID NO: 54
H S¹ D G I F T D S¹ Y S¹ R Y R K Q M A V K K Y L A A V S¹ SEQ ID NO: 55
H s¹ D G I F T D s/S¹ Y S¹ R Y R K Q M A V K K Y L A A V S¹ SEQ ID NO: 56
H S¹ D G I F T D S¹ Y S¹ R Y R K Q M A V K K Y L A A V s/S¹ SEQ ID NO: 57
Mel = melibiose; s = D-Serine; Ñ = L-Nor-Leucine; S* = L-Ser(β-D-Glc);
\check{S} = L-Ser(β-Melibiose); Sar = Sarcosine; a = D-Alanine; βA = β-Alanine;
Dava = Diaminovaleric Acid; B = α-Aminoisobuteric Acid; tL =
L-tert-Leucine; Nva = L-nor-Valine; Å = L-N-Methylalanine; Å′ =
D-N-Methylalanine; X¹⁷ = M or Ñ at 17-position; ¹ = each is optionally
and independently glycosylated with glucose, galactose, melibiose,
xylose, lactose, trehalose, altose, or other monosaccharide or a
disaccharide described herein; and s/S = D- or L-isomer of serine,
respectively. In all cases, at least one of the amino acid residue is
glycosylated.

The ability of glycopeptides of the invention to cross the blood-brain barrier can be readily determined by one skilled in the art having read the present disclosure. Exemplary examples for determining the blood-brain barrier crossing can be found in the Examples section below.

In some embodiments, the in vivo plasma half-life (t_{1/2}) of glycopeptides of the present invention is at least 20 min, typically at least 30 min, often at least 40 min and more often at least 60 min. Alternatively, the half-life of glycopeptides of the invention is at least about 600% or more, typically at least about 1200% or more, often at least about 1400% or more, and more often at least 1700% or more than the same peptide in the absence of the saccharide portion.

Still in other embodiments, the amount of blood-brain barrier crossing by glycopeptides of the invention is increased by at least about 1000%, typically at least about 1200%, often at least about 1500%, and more often at least about 1700% compared to the same peptide in the absence of the saccharide portion.

Some representative agonist data for glycopeptides of SEQ ID NO:4 are provided in Table 2 below.

| | PAC1 | | VPAC1 | | VPAC2 | |
|---|---|---|---|---|---|---|
| | EC₅₀ | E_{Max} | EC₅₀ | E_{Max} | EC₅₀ | E_{Max} |
| Sequence | (nM) | (%) | (nM) | (%) | (nM) | (%) |
| HSDGIFTDSYSRYRKQMAVKKYLAAVL | 0.4, | 100 | 14.8 ± 1.6 | 100 | 0.35 ± 0.16 | 100 |
| | 0.13, | | | | | |
| | 0.34 | | | | | |
| HSDGIFTDSYSRYRKQLAVKKYLAAVL-Ser(Mel) | 0.57 | 99 | 0.55 | 102 | 9.4 | 86 |
| HsDGIFTDSYSRYRKQÑAVKKYLAAVL-Ser(Glc) | 25.5 | 85 | 1.3 | 90 | 241 | 104 |
| HsDGIFTDSYSRYRKQÑAVKKYLAAV-Ser(Glc) | 54.5 | 86 | 4.8 | 90 | 654 | 107 |
| HsDGIFTDSYSRYRKQÑAVKKYLAAL-Ser(Glc)-L | >250 | −79 | 5.9 | 93 | >2500 | −95 |
| HsDGIFADSYSRYRKQÑAVKKYLAAVL-Ser(Glc) | >250 | −71 | 78.8 | 93 | >2500 | −42 |
| HsDGIFADSYSRYRKQÑAVKKYLAAV-Ser(Glc) | NC | NC | 1366 | 85 | NC | NC |
| HsDGIFADSYSRYRKQÑAVKKYLAAV-Ser(Glc)-L | NC | NC | 1623 | 78 | NC | NC |

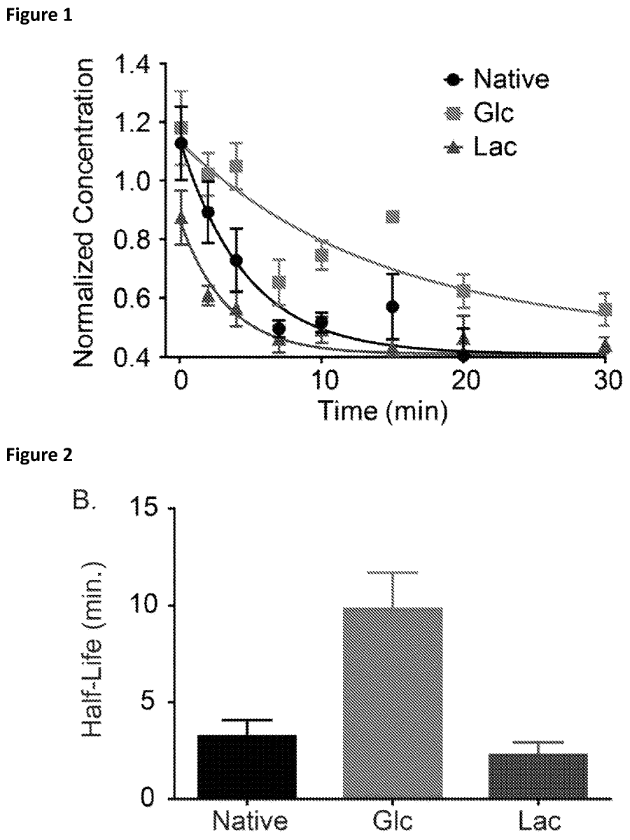

Chemical stability of the glycopeptides in vivo plays an important role in the deliverability of the drugs to the site(s) of action within the brain. It is also important to know what the chemical or metabolic instabilities are in order to inform the drug design process. To this end, stability of PACAP₁₋₂₇; PACAP_{1-27-S-G} and PACAP_{1-27-S-L} was tested. As shown in FIG. 1, PACAP₁₋₂₇ and its glycosylated analogues degraded over 30 min in mouse serum at 37° C. Data were fitted using a single exponential decay model (R²>0.71, in all cases). FIG. 2 shows that Serine glucoside (PACAP_{1-27-S-G}, Glc) showed a significant increase in mouse serum t_{1/2} in vitro compared to the native peptide PACAP₁₋₂₇ and the corresponding lactoside (PACAP_{1-27-S-L}, Lac) when compared using a 1-way analysis of variance (F2=12.91, p=0.0067, Tukey's multiple comparison Native vs Glc, q=5.760 p<0.05, Lac vs Glc, q=6.602 p<0.5).

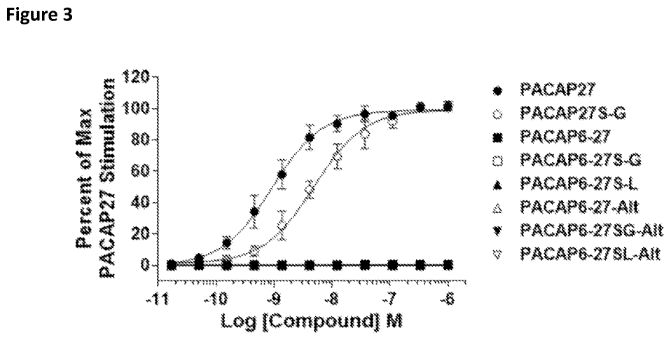

Using CHO cells that express human PAC₁ receptors, PACAP₁₋₂₇, the glucoside PACAP_{27-S-G}, and the truncated putative antagonist PACAP₆₋₂₇ and its derivatives were tested as agonists using FLIPR. It was found that PACAP₁₋₂₇ and PACAP_{1-27-S-G}, the serine glucoside, activated PAC₁ with high potency (0.95±0.4 nM and 5.68±2.3 nM, respectively). See FIG. 3. In addition, the normalized efficacy of the PACAP_{1-27-S-G} glucoside was nearly identical to the native PACAP₁₋₂₇ peptide, at 101.9±1.6%. These findings strongly indicate that glycosylation of PACAP₁₋₂₇ does not significantly alter binding and activation of the PAC₁ receptor, supporting the use of such a glycopeptide for therapeutic purposes. As expected, none of the PACAP₆₋₂₇ derivatives showed agonist activity at concentrations up to 1 μM. See FIG. 3.

PAC₁-CHO calcium flux activation was measured using FLIPR in response to 11 point concentration curves of PACAP₁₋₂₇, the glucoside PACAP_{1-27-S-G}, and the truncated derivatives (putative antagonists) of PACAP₆₋₂₇. Response was measured over 10 minutes, the max-min calculated, and all data was normalized to the maximum response caused by PACAP₁₋₂₇ (100%) and vehicle (0%). The mean±SEM is shown, using the mean value from each independent experiment. N=3 independent experiments performed, 3 variable non-linear curve fit using Prism. PACAP₁₋₂₇ and PACAP_{1-27-S-G} showed potent, efficacious agonist activity.

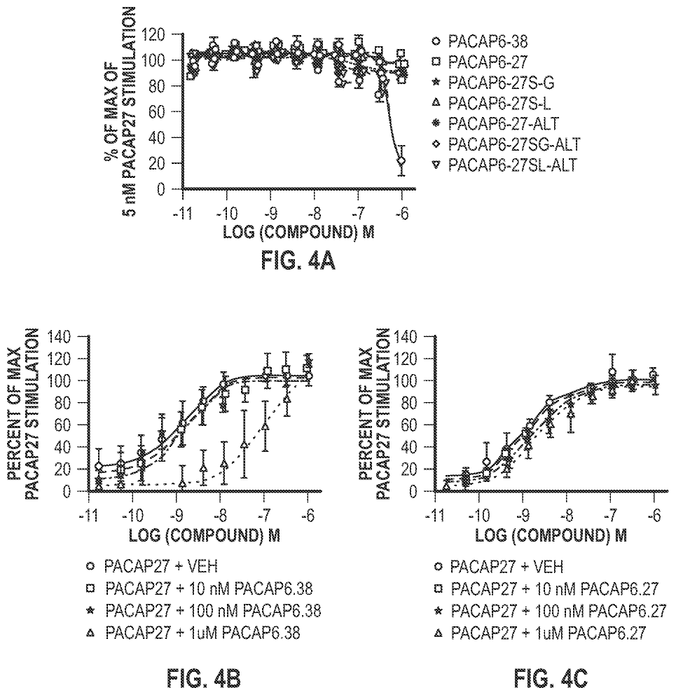

The ability of glycosylated and non-glycosylated PACAP₆₋₂₇ derivatives to block activation of the PAC₁ receptor by PACAP₁₋₂₇ was also tested. A variable concentration mode antagonist assay versus 5 nM of PACAP₁₋₂₇ in the PAC₁-CHO cells was employed using FLIPR. Surprisingly, no significant antagonist activity of PACAP₆₋₂₇ or any derivative at a concentration up to 1 μM was detected. See FIG. 4A-C. When the known antagonist PACAP₆₋₃₈ was tested, only a low potency antagonism was detected (>333 nM). See FIG. 4A. These findings are at odds with a molecular pharmacology study of PACAP₆₋₃₈ and PACAP₆₋₂₇ with reported Ki values of 1.5 and 60 nM, respectively.

The ability of PACAP₆₋₂₇ derivatives to block PACAP₁₋₂₇ induced calcium flux was measured using FLIPR. FIG. 4A shows variable concentration mode antagonist experiments. Concentration curves of PACAP₆₋₃₈, PACAP₆₋₂₇, and PACAP₆₋₂₇ derivatives were added to the cells for 2 minutes, followed by 5 nM of PACAP₁₋₂₇. The max-min response was determined, and normalized to the stimulation caused by 5 nM PACAP₁₋₂₇ (100%) and vehicle (0%). Only PACAP₆₋₃₈ showed antagonism, but it is low potency. FIG. 4B shows fixed concentration mode experiments with PACAP₆₋₃₈. Fixed concentrations of PACAP₆₋₃₈ was added to cells for 2 minutes, followed by concentration curves of PACAP₁₋₂₇. The max-min response was determined, and normalized to the max response of the PACAP₁₋₂₇ curve without antagonist present (100%) and vehicle (0%). PACAP₆₋₃₈ shifted the curve only at the highest concentration (1 μM). FIG. 4C shows fixed concentration mode experiments with PACAP₆₋₂₇, performed as in FIG. 4B N=3 independent experiments. PACAP₆₋₂₇ showed no detectable shifts in the agonist curves.

Fixed concentration antagonist mode experiments was performed with the peptides PACAP₆₋₂₇ and PACAP₆₋₃₈. It was found that PACAP₆₋₂₇ caused no shift in the agonist curves, while PACAP₆₋₃₈ induced a shift only at 1 μM. See FIGS. 4B and 4C. This resulted in a pA2 value of 200.6±55.4 nM for PACAP₆38, well above the 1.5 nM value previously reported. Notably, PACAP₆₋₃₈ also showed a Schild Slope of 2.0±0.1. A Schild Slope of 1 fits the assumptions of the model, while a slope above 1 suggests that the compound is more effective than would be expected for competitive antagonism. Without being bound by any theory, it is believed that this result may be due to the short incubation times in the FLIPR assay, which might not be long enough to allow the system to reach equilibrium, thus leading to a relatively high Schild Slope. Alternatively, PACAP₆₋₃₈ may function by a different mechanism, e.g., binding to VPAC_{1/2}.

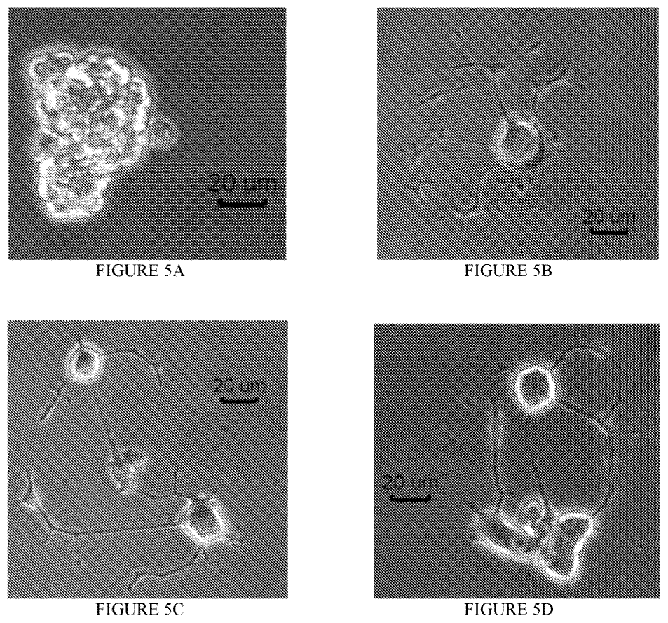

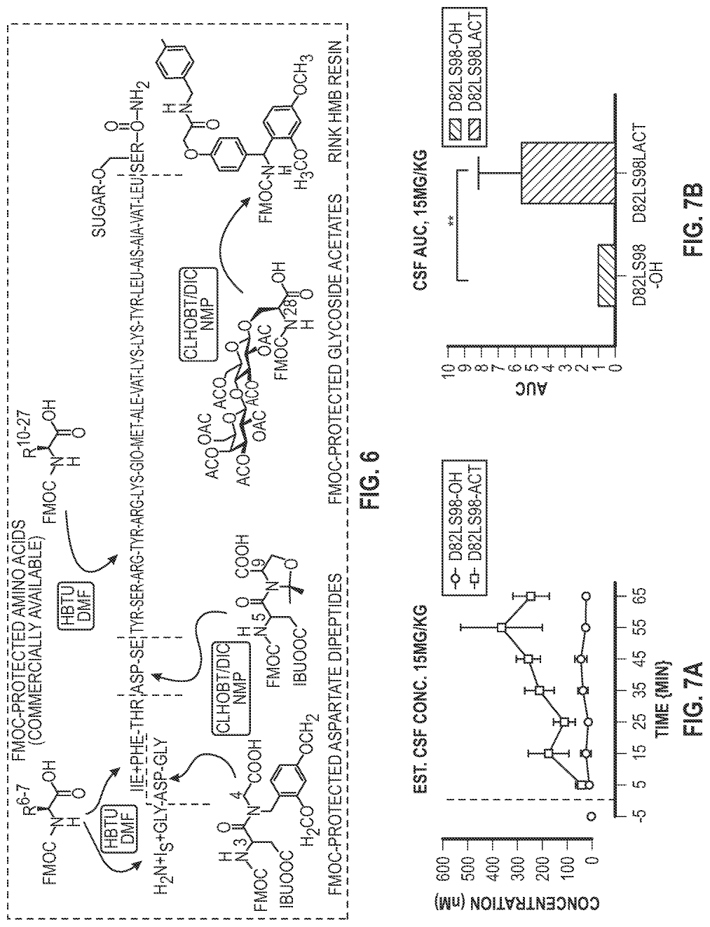

PC12 cells are non-adherent cells, and in spite of using the poly-D-Lysine coated plates, the majority of the cells remained suspended. During the media exchange many of the cells were removed with the spent media. The remaining cells could be visually evaluated for qualitative morphological changes at the end of the treatment period, but meaningful cell quantification could not be done reliably using this approach. It was found that glucoside and lactoside PACAP₁₋₂₇ derivative treatment produced neurite outgrowth and arborization when compared to vehicle treated cells. FIGS. 5A-D. Qualitatively, it appeared that the arborization caused by PACAP₁₋₂₇ may be more extensive than that caused by the glucoside and lactoside derivatives, but again this could not be quantified. In any case, both PACAP₁₋₂₇ and the derivatives induced neurite outgrowth, suggesting native PAC₁ agonist activity.

FIGS. 5A-5D show PC12 Cell Morphology after Vehicle vs PACAP Treatment (100 nM). FIG. 5A: diluent only; 5B: PACAP₁₋₂₇; 5C: PACAP_{1-27-S-G}; 5D: PACAP_{1-27-S-L}. The cell body volumes all showed increases when treated with each of the PACAP derivatives. In all cases the process outgrowths on the treated cells were greater than 2× the cell body width

Endogenous PACAP peptides occur as C-terminal peptide amides that have either 27 (10%) or 38 (90%) amino acid residues, and are typically regarded as PAC₁ agonists in assays using intact tissue or in cell culture. For the present studies, a separate CHO cell line expressing the PAC₁ receptor individually was developed. Use of solid-phase peptide synthesis allowed a wide different glycopeptides as illustrated in Table 1 above. As can be seen, glycopeptides of the invention retained their agonist activity on PC12 cell cultures (FIGS. 5A-5D) and in the quantitative CHO cell assay (FIG. 3). In addition, glycopeptides of the invention had a higher half-life in mouse serum compared to a corresponding non-glycosylated peptides. More significantly, experiments showed glycopeptides of the invention can cross the blood-brain barrier in mice. In use, an effective amount of glycopeptides of the present invention can be administered to a patient in need of treatment in a therapeutically effective unit dose delivery amount of between about 0.1 and 10 milligrams per kilo, typically 1-2 doses per day, or even less frequently. The glycopeptides of the invention may be delivered in a pharmaceutically acceptable carrier.

Pharmaceutical formulations and pharmaceutical compositions are well known in the art, and can be found, for example, in Remington's Pharmaceutical Sciences, Mack Publishing Co., Easton, Pa., USA, which is hereby incorporated by reference in its entirety. Any formulations described therein or otherwise known in the art are embraced by embodiments of the disclosure.

Pharmaceutical excipients are well known in the art and include, but are not limited to, saccharides such as, for example, lactose or sucrose, mannitol or sorbitol, cellulose preparations, calcium phosphates such as tricalcium phosphate or calcium hydrogen phosphate, as well as binders, such as, starch paste such as, for example, maize starch, wheat starch, rice starch, potato starch, gelatin, tragacanth, methyl cellulose, hydroxypropylmethylcellulose, sodium carboxymethylcellulose, polyvinyl pyrrolidone or combinations thereof.

In particular embodiments, pharmaceutical formulations may include the active compound described and embodied above, a pharmaceutically acceptable carrier or excipient and any number of additional or auxiliary components known in the pharmaceutical arts such as, for example, binders, fillers, disintegrating agents, sweeteners, wetting agents, colorants, sustained release agents, and the like, and in certain embodiments, the pharmaceutical composition may include one or more secondary active agents. Disintegrating agents, such as starches as described above, carboxymethyl-starch, cross-linked polyvinyl pyrrolidone, agar, alginic acid or a salt thereof, such as sodium alginate and combinations thereof. Auxiliary agents may include, for example, flow-regulating agents and lubricants, such as silica, talc, stearic acid or salts thereof, such as magnesium stearate or calcium stearate, polyethylene glycol and combinations thereof. In certain embodiments, dragee cores may be prepared with suitable coatings that are resistant to gastric juices, such as concentrated saccharide solutions, which may contain, for example, gum arabic, talc, polyvinyl pyrrolidone, polyethylene glycol, titanium dioxide, lacquer solutions and suitable organic solvents or solvent mixtures and combinations thereof. In order to produce coatings resistant to gastric juices, solutions of suitable cellulose preparations, such as acetylcellulose phthalate or hydroxypropylmethyl-cellulose phthalate may also be used. In still other embodiments, dye or pigments may be added to the tablets or dragee coatings, for example, for identification or in order to characterize combinations of active compound doses.

Pharmaceutical compositions of the disclosure can be administered to any animal, and in particular, any mammal, that may experience a beneficial effect as a result of being administered a compound of the disclosure including, but not limited to, humans, canines, felines, livestock, horses, cattle, sheep, and the like. The dosage or amount of at least one compound according to the disclosure provided pharmaceutical compositions of embodiments may vary and may depend, for example, on the use of the pharmaceutical composition, the mode of administration or delivery of the pharmaceutical composition, the disease indication being treated, the age, health, weight, etc. of the recipient, concurrent treatment, if any, frequency of treatment, and the nature of the effect desired and so on. Various embodiments of the disclosure include pharmaceutical compositions that include one or more compounds of the disclosure in an amount sufficient to treat or prevent diseases such as, for example, cancer. An effective amount of the one or more compounds may vary and may be, for example, from about 0.1 to 10 milligrams per kilo, typically 1-2 doses per day.

The pharmaceutical compositions of the disclosure can be administered by any means that achieve their intended purpose. For example, routes of administration encompassed by the disclosure include, but are not limited to, subcutaneous, intravenous, intramuscular, intraperitoneal, buccal, or ocular routes, rectally, parenterally, intrasystemically, intravaginally, topically (as by powders, ointments, drops or transdermal patch), oral, deep lung, intranasal administration or nasal spray (nebulization) are contemplated in combination with the above described compositions.

Embodiments of the disclosure also include methods for preparing pharmaceutical compositions as described above by, for example, conventional mixing, granulating, dragee-making, dissolving, lyophilizing processes and the like. For example, pharmaceutical compositions for oral use can be obtained by combining the one or more active compounds with one or more solid excipients and, optionally, grinding the mixture.

Suitable auxiliaries may then be added and the mixture may be processed to form granules which may be used to form tablets or dragee cores. Other pharmaceutical solid preparations include push-fit capsules containing granules of one or more compound of the disclosure that can, in some embodiments, be mixed, for example, with fillers, binders, lubricants, stearate, stabilizers or combinations thereof. Push-fit capsules are well known and may be made of gelatin alone or gelatin in combination with one or more plasticizer such as glycerol or sorbitol to form a soft capsule. In embodiments in which soft capsules are utilized, compounds of the disclosure may be dissolved or suspended in one or more suitable liquids, such as, fatty oils or liquid paraffin and, in some cases, one or more stabilizers.

Liquid dosage formulations suitable for oral administration are also encompassed by embodiments of the disclosure. Such embodiments, may include one or more compounds of the disclosure in pharmaceutically acceptable emulsions, solutions, suspensions, syrups, and elixirs that may contain, for example, one or more inert diluents commonly used in the art such as, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (for example, cottonseed, groundnut, corn, germ, olive, castor, and sesame oils), glycerol, fatty acid derivatives of glycerol (for example, labrasol), tetrahydrofurfuryl alcohol, polyethylene glycols and fatty acid esters of sorbitan, and mixtures thereof. Suspensions may further contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations for parenteral administration may include one or more compounds of the disclosure in water-soluble form, for example, water-soluble salts, alkaline solutions, and cyclodextrin inclusion complexes in a physiologically acceptable diluent which may be administered by injection. Physiologically acceptable diluent of such embodiments, may include, for example, sterile liquids such as water, saline, aqueous dextrose, other pharmaceutically acceptable sugar solutions; alcohols such as ethanol, isopropanol or hexadecyl alcohol; glycols such as propylene glycol or polyethylene glycol; glycerol ketals such as 2,2-dimethyl-1,3-dioxolane-4-methanol; ethers such as poly(ethyleneglycol)400; pharmaceutically acceptable oils such as fatty acid, fatty acid ester or glyceride, or an acetylated fatty acid glyceride. In some embodiments, formulations suitable for parenteral administration may additionally include one or more pharmaceutically acceptable surfactants, such as a soap or detergent; suspending agent such as pectin, carbomers, methylcellulose, hydroxypropylmethylcellulose, or carboxymethylcellulose; an emulsifying agent; pharmaceutically acceptable adjuvants or combinations thereof. Additional pharmaceutically acceptable oils which may be useful in such formulations include those of petroleum, animal, vegetable or synthetic origin including, but not limited to, peanut oil, soybean oil, sesame oil, cottonseed oil, olive oil, sunflower oil, petrolatum, and mineral oil; fatty acids such as oleic acid, stearic acid, and isostearic acid; and fatty acid esters such as ethyl oleate and isopropyl myristate. Additional suitable detergents include, for example, fatty acid alkali metal, ammonium, and triethanolamine salts; cationic detergents such as dimethyl dialkyl ammonium halides, alkyl pyridinium halides, and alkylamine acetates; and anionic detergents, such as alkyl, aryl, and olefin sulfonates, alkyl, olefin, ether and monoglyceride sulfates, and sulfosuccinates. In some embodiments, non-ionic detergents including, but not limited to, fatty amine oxides, fatty acid alkanolamides and polyoxyethylenepolypropylene copolymers or amphoteric detergents such as alkyl-β-aminopropionates and 2-alkylimidazoline quaternary salts, and mixtures thereof may be useful in parenteral formulations of the disclosure.

Pharmaceutical compositions for parenteral administration may contain from about 0.5 to about 25% by weight of one or more of the compounds of the disclosure and from about 0.05% to about 5% suspending agent in an isotonic medium. In various embodiments, the injectable solution should be sterile and should be fluid to the extent that it can be easily loaded into a syringe. In addition, injectable pharmaceutical compositions may be stable under the conditions of manufacture and storage and may be preserved against the contaminating action of microorganisms such as bacteria and fungi.

Topical administration includes administration to the skin or mucosa, including surfaces of the lung and eye. Compositions for topical administration, may be prepared as a dry powder which may be pressurized or non-pressurized. In non-pressurized powder compositions, the active ingredients in admixture are prepared as a finely divided powder. In such embodiments, at least 95% by weight of the particles of the admixture may have an effective particle size in the range of 0.01 to 10 micrometers. In some embodiments, the finely divided admixture powder may be additionally mixed with an inert carrier such as a sugar having a larger particle size, for example, of up to 100 micrometers in diameter. Alternatively, the composition may be pressurized using a compressed gas, such as nitrogen or a liquefied gas propellant. In embodiments, in which a liquefied propellant medium is used, the propellant may be chosen such that the compound and/or an admixture including the compound do not dissolve in the propellant to any substantial extent. In some embodiments, a pressurized form of the composition may also contain a surface-active agent. The surface-active agent may be a liquid or solid non-ionic surface-active agent or may be a solid anionic surface-active agent, which in certain embodiments, may be in the form of a sodium salt.

Compositions for rectal administration may be prepared by mixing the compounds or compositions of the disclosure with suitable non-irritating excipients or carriers such as for example, cocoa butter, polyethylene glycol or a suppository wax. Such carriers may be solid at room temperature but liquid at body temperature and therefore melt in the rectum and release the drugs.

In still other embodiments, the compounds or compositions of the disclosure can be administered in the form of liposomes. Liposomes are generally derived from phospholipids or other lipid substances that form mono- or multi-lamellar hydrated liquid crystals when dispersed in an aqueous medium. Any non-toxic, physiologically acceptable and metabolizable lipid capable of forming liposomes can be used, and in particular embodiments, the lipids utilized may be natural and/or synthetic phospholipids and phosphatidyl cholines (lecithins). Methods to form liposomes are known in the art (see, for example, Prescott, Ed., Meth. Cell Biol. 14:33 (1976), which is hereby incorporated by reference in its entirety). Compositions including one or more compounds of the disclosure in liposome form can contain, for example, stabilizers, preservatives, excipients and the like.

In general, methods of embodiments of the disclosure may include the step of administering or providing an “effective amount” or a “therapeutically effective amount” of a compound or composition of the disclosure to an individual. In such embodiments, an effective amount of the compounds of the disclosure may be any amount that produces the desired effect. As described above, this amount may vary depending on, for example, the circumstances under which the compound or composition is administered (e.g., to incite treatment or prophylactically), the type of individual, the size, health, etc. of the individual and so on. The dosage may further vary based on the severity of the condition. For example, a higher dose may be administered to treat an individual with a well-developed metastatic condition, compared to the amount used to prevent a subject from developing the metastatic condition. Those skilled in the art can discern the proper dosage based on such factors. For example, in some embodiments, the dosage may be within the range of about 0.01 mg/kg body weight to about 10 mg/kg body weight.

The administration schedule may also vary. For example, in some embodiments, the compounds or compositions of the disclosure may be administered in a single dose once per day or once per week. In other embodiments, the compounds or compositions of the disclosure may be administered in one or two or more doses per day. For example, in one embodiment, an effective amount for a single day may be divided into separate dosages that may contain the same or a different amount of the compound or composition and may be administered several times throughout a single day. Without wishing to be bound by theory, the dosage per administration and frequency of administration may depend, for example, on the specific compound or composition used, the condition being treated, the severity of the condition being treated, and the age, weight, and general physical condition of the individual to which the compound or composition is administered and other medications which the individual may be taking. In another exemplary embodiment, treatment may be initiated with smaller dosages that are less than the optimum dose of the compound, and the dosage may be increased incrementally until a more optimum dosage is achieved.

In each of the embodiments above, the compound administered can be provided as a pharmaceutical composition including compound as described above and a pharmaceutically acceptable excipient or a pure form of the compound may be administered.

In additional embodiments, the compound or composition of the disclosure may be used alone or in combination with one or more additional agents. For example, in some embodiments, a compound or composition of disclosure may be formulated with one or more additional anti-cancer agents or combinations thereof such that the pharmaceutical composition obtained including the compound or composition of the disclosure and the one or more additional agents can be delivered to an individual in a single dose. In other embodiments, the compound or composition of the disclosure may be formulated as a separate pharmaceutical composition that is delivered in a separate dose from pharmaceutical compositions including the one or more additional agents. In such embodiments, two or more pharmaceutical compositions may be administered to deliver effective amounts of a compound or composition of the disclosure and the one or more additional agents.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1 Methods and Materials

Glycopeptides of the invention were prepared using one of the following procedures.

General Method I. The C-terminal amino acids were loaded onto Fmoc-Rink resin (Advanced ChemTech, Louisville, KY, USA) at 0.1 mmol/g resin loading in 25 mL fritted syringes and dimethylformamide (˜5 mL per gram resin) was added. The mixture was stirred at RT for two minutes (x2). A solution of 2% DBU and 3% piperidine in DMF (v:v) was added and the mixture was agitated for 5 minutes, refreshed, and agitated for an additional 10 minutes. The resin was washed with DMF (x5), and finally with N-methylpyrrolidine (NMP). In a separate vial, a solution of Fmoc-β-OGlc(OAc)₄-Ser-OH (0.12 mmol) and HOBt⋅H₂O (0.13 mmol) in 5 mL NMP was prepared. To this solution was added DIC (0.26 mmol) and the mixture was stirred for 5 minutes. The resulting solution was added to the resin and agitated for 10 minutes. Next, the syringe was placed in a microwave oven (Emerson 900 W Microwave—MW9338SB) and irradiated at power level 1 for 10 minutes. The syringe was then agitated at RT for an additional 30 minutes. The resin was washed with NMP (x1), DMF (x5), and CH₂Cl₂ (x5), and dried in vacuo overnight.

General Method II: Glycopeptides were assembled on a Prelude® Peptide Synthesizer (Protein Technologies, Inc., Tucson, AZ, USA) using the following procedure.

Rink resin (100 mg) was placed into the fritted reaction vessels. Amino acids were dissolved in DMF at 250 mM concentration, HATU at 375 mM, and TMP at 3M. The following steps were performed for coupling: DMF Top Wash (1.5 mL, 2 min mix and drain; x6), Deprotection (2% DBU/3% piperidine in DMF; 1.5 mL, 4 min mix and drain; 8 min mix and drain), DMF Top Wash (1.5 ml, 2 min mix and drain; x5), Amino Acid Building Block (0.950 mL, 30 sec mix), Activator 1 (HATU, 0.650 mL, 30 sec mix), Base (TMP, 0.300 mL, 35 min mix and drain), DMF Top Wash (1.5 mL, 2 min mix and drain; x2). After coupling aspartic acid D7, the deprotection solution was changed to 0.1 M HOBt⋅H₂O/5% piperazine in DMF to minimize aspartimide formation.

Cleavage of the glycopeptides from the resin. The glycopeptides prepared using General Method I or II were cleaved from the resin using a mixture of trifluoroacetic acid, triethylsilane, water, methylene chloride and methoxyphenyl (9:0.3:0.2:1:0.05 by volume). Briefly, the mixture was agitated at room temperature (RT) for 2 hours. The resulting solutions were transferred to 15 mL centrifuge tubes, evaporated under argon, precipitated in ice-cold Et₂O, decanted, and rewashed with Et₂O, then dissolved in H₂O and lyophilized to afford the crude material as fluffy white solids.

Purification of the crude glycopeptides was accomplished by Reversed Phase HPLC (RP-HPLC) with a preparative RP (C-18) Phenomenex (250×22 mm) column using a CH₃CN—H₂O gradient solvent system containing 0.1% trifluoroacetic acid. Homogeneity of the purified glycopeptides was confirmed by analytical RP-HPLC and high resolution mass spectrometry.

### Binding Assay.

Binding assay to determine PAC1, VPAC1, and VPAC2 agonist activity can be performed as described by Doan et al. in Biochem. Pharmalcol., 2011, 81, pp. 552-561. Briefly, the binding assay procedure is as follows: Acetylated PACAP₂₇ was radio-iodinated using the chloramine-T technique (Hunter WMGF, Nature, 1962,194, pp. 495-6) and purified on a Sep-Pak C18 cartridge (Waters, Milford, MA, USA). CHO cells expressing one of the PACAP-related receptors were seeded at a density of 125,000 cells per well in 24-well plates. After 24 h, the culture medium was removed, and cells were first incubated at room temperature for 10 min in binding buffer (0.1% BSA, 25 mM Tris-HCl, 25 mM MgCl₂, and 5 mg/L bacitracin, pH 7.4) and then exposed to increasing peptide concentrations in the presence of 0.05 nM ¹²⁵I—Ac-PACAP27. After 2 h at room temperature, cells were washed twice with binding buffer, lysed (0.1 M NaOH), and the cell-bound radioactivity was quantified using a g-counter (1470 Automatic Gamma Counter, Perkin Elmer). Results were expressed as the percentage of the specific binding of ¹²⁵I—Ac-PACAP27 obtained in the absence of competitive ligands. Nonspecific binding was determined in the presence of 10 mM PACAP38 and averaged at 5-10% of total binding.

### Biological Assay.

The neuroprotective effects of the PAC₁ agonist and the anti-inflammatory effects of the VPAC₁ antagonist in cell culture models were tested. In addition, stability of peptides and BBB penetration in vivo were also tested. These tests were used to identify peptides for therapeutic candidates in vivo in preclinical models of ALS and PD.

The overall process was as follows: Flow-injection tandem mass spectrometry (FI-MSⁿ) was used to observe the degradation of the peptides and glycopeptides with a Thermo LCQ with electrospray ionization (ESI). The technique involved injection of a sample bolus of material in mouse serum via a six port valve with fluid flow delivered via a syringe pump, and subsequent electrospray ionization (ESI) followed by mass spectral analysis. Samples were diluted to a concentration of ˜5 μM of each PACAP analogue, and were incubated at 37° C. for times varying from 1 to 60 minutes. After samples had been incubated for the prescribed amount of time they were prepared for mass spectrometry analysis by withdrawing 10 microliters of solution and spiking with 1 microliter of a 10 μM solution of peptide internal standard (angiotensin II) in 50% acetic acid and subjecting them to a standard C18 zip tip desalting. These solutions, once eluted from the zip tip were diluted to 100 μL in 50:50 acetonitrile/water with 0.1% formic acid. Tandem mass spectrometry analysis (MS³) was conducted to yield specific, quantitative signals proportional to the amount of PACAP analogue at each time point. This technique was also used with microdiasylate samples from a mouse after i.p. administration of PACAP1-27-S-G.

A custom DNA clone of the human PAC1 gene with 3 hemagglutinin (HA) tags inserted 3′ to the signal peptide sequence (to avoid proteolytic loss) was obtained from Genecopoeia (Rockville, MD). The construct was electroporated into Chinese Hamster Ovary (CHO) cells, and selected for with 500 μg/mL of G418. The resulting population was screened for high expressing clones, and one such clone selected for further analysis. The clonal cell line (PAC₁-CHO) displayed high receptor expression by immunocytochemistry and Western blot, and showed selective activation of signaling in response to PACAP₁₋₂₇. This cell line was used for all molecular pharmacology experiments. The cells were maintained in DMEM/F12 with 10% heat-inactivated FBS, 1× penicillin/streptomycin, and 500 μg/mL G418, at 37° C. and 5% CO₂.

All molecular pharmacology experiments were carried out using a FLIPR Tetra from Molecular Devices (Sunnyvale, CA), set to image calcium flux using the manufacturer's recommended settings and protocols. The day before an experiment, the PAC₁-CHO cells were split into 384 well black walled, clear bottom microplates, 10,000 cells per well. The cells were recovered overnight in growth medium (as above). The next day, the growth medium was replaced with Calcium 6 dye (Molecular Devices) using the manufacturer recommended buffer with 2.5 mM probenecid. The cells were incubated for 2 hours in the culture incubator, and removed during the last 15 minutes to allow equilibration to room temperature. Compound as indicated below was added to the cells using a 384 tip block, with real time monitoring before, during, and 15 minutes after compound addition. The resulting calcium flux was recorded, and the maximum-minimum response over the entire observation time calculated and reported as the mean±SEM (4 wells per point).

For agonist mode experiments, compound was added in an 11 point concentration curve, with a vehicle control (buffer). The resulting response was normalized to the stimulation caused by PACAP₁₋₂₇ (100%) and vehicle (0%). The response was analyzed using a 3 variable non-linear curve fit, and the EC₅₀ (nM) and E_{Max} (%) calculated and reported (Prism, GraphPad, La Jolla, CA).

For antagonist mode experiments, a concentration curve (variable concentration mode) or fixed amount (fixed concentration mode) of antagonist was added to the cells, and allowed to equilibrate for 2 minutes. Then, either a 5 nM fixed concentration (variable concentration mode) or an 11 point concentration curve (fixed concentration mode) of PACAP₁₋₂₇ was added to the cells, and the max-min response recorded as above. For variable concentration mode experiments, the data was normalized to the stimulation caused by 5 nM PACAP₁₋₂₇ (100%) and vehicle (0%), and analyzed with a 3 variable non-linear curve fit, with the IC₅₀ (nM) and I_{Max}(%) calculated and reported (Prism). For the fixed concentration mode experiments, each curve was normalized to the maximum stimulation caused by PACAP₁₋₂₇ with no antagonist present (100%) and vehicle (0%). The resulting data was analyzed using a Gaddum/Schild EC₅₀ shift model, (Schild, 1957, Gaddum, 1957) designed to analyze competitive antagonism. (Lazareno and Birdsall, 1993) The data output was the pA2 (nM) and the Schild Slope, a measure of how closely the experimental data fits the operational model of competitive antagonism (Prism). For all analyses, each independent experiment performed in quadruplicate is considered to be a sample size of 1. The pharmacology values are calculated separately from each experiment, then combined and reported as the mean±SEM for the entire set of experiments.

The PC12 cells were cultured in RPMI containing 5% heat inactivated fetal bovine serum and 10% horse serum in the presence of 100 units/mL penicillin and 100 microgram/mL streptomycin. The cells were plated on poly-D-Lysine coated 6-well tissue culture plates at a density of 150,000 cells per well in 2 mL media. After 48 hours at 37° C. in 5% CO₂ atmosphere, media exchange was performed and plates were dosed, using the peptide diluent (water) for the control samples. PACAP₁₋₂₇, PACAP_{1-27-S-G}, and PACAP_{1-27-S-L} were used to screen for PAC₁ receptor activation. Four groups of cells were used; one control group (diluent treated) and three treatment groups, each treatment group was exposed to 100 nM concentrations of PACAP₁₋₂₇, PACAP₁₋₂₇-S-G, or PACAP₁₋₂₇-s-L. All groups were run in triplicate. Cell images of each treatment group were captured and compared to the control cells to screen for differentiation and cell body volume increases. Cells having neurite-like process outgrowth were noted and photographed. The neurite-like outgrowth was deemed positive if its length was at least two times the width of the cell body.

### Example 2 Synthesis of PACAP Glycopeptides

A modest library of 47 glycopeptide analogues of PACAP were successfully synthesized, purified, and characterized by HPLC and MS. The carbohydrate moieties were introduced “pre-translationally” by incorporation of Fmoc-Serine glycoside building blocks that were prepared using “minimally competent” InBr₃ catalysis (Mitchell et al., 200; Lefever et al., 2012). PACAP is considered a “difficult peptide sequence” due to its length and presence of two dipeptide motifs within its structure that are prone to aspartimide formation (Dölling et al., 1994; Lauer et al., 1995; Subirós-Funosas et al., 2011; Paradís-Bas et al., 2016; Samson et al., 2019). Thus, the use of a single coupling protocol and standard Fmoc amino acid building blocks typically results in low yields and purity. We addressed these problems by utilizing several different coupling protocols for the PACAP derivatives (FIG. 4). The glycosidic amino acid at the C-terminus was coupled to the resin using 6-Cl-HOBt and an equimolar amount of DIC in NMP. The residues Tyr¹⁰ through Leu²⁷ were coupled using a standard HBTU/N-Methylmorpholine protocol. Aspartimide formation was suppressed by incorporating Fmoc-protected diamino acid building blocks containing either pseudoproline or dimethoxybenzyl (DMB)-containing motifs (FIG. 4) (Haack and Mutter, 1992; Wöhr Mutter 1995;Wöhr et al., 1996; Sampson et al., 1999; White et al., 2004; Cardona et al., 2008). The dipeptides Asp⁸-Ser⁹ and Asp³-Gly⁴ were coupled using 6-C1-HOBt/DIC in NMP. The lipophilic tripeptide motif consisting of Ile⁵, Phe⁶, and Thr⁷ residues were coupled using HATU and 2,4,6-collidine in DMF. The remaining amino acids (His¹ and Ser²) were coupled using the 6-Cl-HOBt/DIC protocol. Acetate protection on the carbohydrate hydroxyls were cleanly removed on-resin using a 50% solution of NH₂NH₂⋅H₂O in NMP. At this stage the peptides were cleaved from the resin with a standard cleavage cocktail [TFA/DCM/H₂O/HSiEt₃/anisole (90:10:2:3:0.5)], precipitated in cold diethyl ether, and purified using RP-HPLC (see section 2). Satisfyingly, this approach resulted in improved purity and yield compared to our initial studies. Table 3 lists the synthesized peptides.

TABLE 3
List of synthesized PACAP peptides

| Compound | Structure |
|---|---|
| 1st series | |
| 2L372-1 | HSDGIFTDSY₁₀SRYRKOLSVK₂₀KYLAAVL-CONH [symbol] |
| 2L372-2 | HSDGIFTDSY₁₀SRYRKOLSVK₂₀KYLAAVL-CONH [symbol] |
| 2L [symbol] 72-3 | HsDGIFTDSY₁₀SRYRKOLSVK₂₀KYLAAVL-CONH [symbol] |
| 2L [symbol] | HSDGIFTDSY₁₀SRYRKOLSVK₂₀KYLAAVL-Ser(Glc)-CONH [symbol] |
| 2L [symbol] | HSDGIFTDSY₁₀SRYRKOLSVK₂₀KYLAAVL-Ser(Lac [symbol] )-CONH [symbol] |
| 2L3 [symbol] | HSDGIFTDSY₁₀SRYRKOLSVK₂₀KYLAAVL-Ser( [symbol] )-CONH [symbol] |
| 2L [symbol] | HSDGIFTDSY₁₀SRYRKOLSVK₂₀KYLAAVL-Ser( [symbol] )-CONH [symbol] |
| 2nd Series | |
| CRA3000 | HsDGIFTDSY₁₀SRYRKO- [symbol] KYLAAVL-Ser(Glc)-CONH [symbol] |
| CRA3001 | HsDGIFTDSY₁₀SRYRKO- [symbol] KYLAAV-Ser(Glc)-CONH [symbol] |
| CRA3002 | HsDGIFTDSY₁₀SRYRKO- [symbol] KYLAA-Ser(Glc)-L-CONH [symbol] |
| CRA3003 | HsDGIFTDSY₁₀SRYRKO- [symbol] KYLAAVL-Ser(Glc)-CONH [symbol] |
| CRA3004 | HsDGIFTDSY₁₀SRYRKO- [symbol] KYLAAV-Ser(Glc)-L-CONH [symbol] |
| CRA3005 | HsDGIFTDSY₁₀SRYRKO- [symbol] KYLAA-Ser(Glc)-CONH [symbol] |
| 2L [symbol] | HSDGIFTDSY₁₀SRYRKOLAVK [symbol] KYLAAVL-Ser( [symbol] )-CONH [symbol] |
| 2L [symbol] | HSDGIFTDSY₁₀SRYRKOLAVK [symbol] KYLAAV [symbol] L-Ser( [symbol] )-CONH [symbol] |
| 2L [symbol] | HSDGIFTDSY₁₀SRYRKOLAV [symbol] KYLAAV [symbol] L-Ser( [symbol] )-CONH [symbol] |
| 3rd Series | |
| CRA3006 | HsD-GABA-IFTDSY₁₀SRYRKO-N [symbol] -AVK [symbol] KYLAAVL-Ser(Glc)-CONH [symbol] |
| CRA3007 | HsD-Ser-IFTDSY₁₀SRYRKO-N [symbol] -AVK [symbol] KYLAAVL-Ser(Glc)-CONH [symbol] |
| CRA3008 | HsD- [symbol] -IFTDSY₁₀SRYRKO-N [symbol] -AVK [symbol] KYLAAVL-Ser(Glc)-CONH [symbol] |
| CRA3009 | HsD-DAVA-IFTDSY₁₀SRYRKO-N [symbol] -AVK [symbol] KVLAAVL-Ser(Glc)-CONH [symbol] |
| CRA3010 | HsDAIFTDSY₁₀SRYRKO-N [symbol] -AVK [symbol] KYLAAVL-Ser(Glc)-CONH [symbol] |
| CRA3011 | HsD-A [symbol] -IFTDSY SRYRKO-N [symbol] -AVK [symbol] KYLAAVL-Ser(Glc)-CONH [symbol] |
| CRA3012 | HsDG [symbol] FTDST₁₀SRYRKO-N [symbol] -AVK [symbol] KYLAAVL-Ser(Glc)-CONH [symbol] |
| CRA3013 | HsDG [symbol] FTDSY₁₀SRYRKO-N [symbol] -AVK [symbol] KYLAAVL-Ser(Glc)-CONH [symbol] |
| CRA3014 | HsDGAFTDSY₁₀SRYRKO-N [symbol] -AVK [symbol] KYLAAVL-Ser(Glc)-CONH [symbol] |
| CRA3015 | HsDG-T [symbol] -FTDSY₁₀SRYRKO-N [symbol] -AVK [symbol] KYLAAVL-Ser(Glc)-CONH [symbol] |
| CRA3016 | HsDG-N [symbol] -FTDSY₁₀SRYRKO-N [symbol] -AVK [symbol] KYLAAVL-Ser(Glc)-CONH [symbol] |
| CRA3017 | HsDG- [symbol] -FTDSY₁₀SRYRKO-N [symbol] -AVK [symbol] KYLAAVL-Ser(Glc)-CONH [symbol] |
| 2L [symbol] 132 | HSDGIFTSDY₁₀SRYRKOLAVK₂₀KYLAAVLS(Lac)-DC [symbol] H [symbol] |
| 4th Series | |
| Ao-2L [symbol] 132 | Ao-HSDGIFTDSY₁₀SRYRKOLAVK₂₀KYLAAVLS(Lac)-CONH [symbol] |
| Ao-CRA3000 | Ao-HsDGIFTDSY₁₀SRYRKO-N [symbol] -AVK [symbol] KYLAAVL-Ser(Glc)-CONH [symbol] |
| CRA3000-Lac | HsDGIFTDSY₁₀SRYRKO-N [symbol] -AVK [symbol] KYLAAVLSer (Lac)-CONH [symbol] |
| Ao-CR [symbol] -La [symbol] . | Ao-HsDGIFTDSY₁₀SRYRKO-N [symbol] -AVK [symbol] KYLAAVLSer ( [symbol] )-CONH [symbol] |
| CRA3018 | HsD-Ser-N [symbol] -FTDSY₁₀SSRYRKO-N [symbol] -AVK [symbol] KYLAAVL-Ser( [symbol] )-CONH [symbol] |
| Ao-CRA3018 | Ao-HsD-Ser-N [symbol] -FTDSY₁₀SRYRKO-N [symbol] -AVK [symbol] KYLAAVL-Ser(Glc)-CONH [symbol] |
| CRA3019 | HsDG-D-N [symbol] -FTDSY₁₀SRYRKO-N [symbol] -AVK₂₀KYLAAVL-Ser(Glc)-CONH [symbol] |
| Ao-CRA3019 | Ao-HsDG-D-N [symbol] -FTDSY₁₀SRYRKO-N [symbol] -AVK [symbol] KYLAAVL-Ser( [symbol] )-CONH [symbol] |
| CRA3020 | HsDG-D-N [symbol] -FTDSY₁₀SRYRKO-N [symbol] -AVK [symbol] YLAAVL-Ser( [symbol] )-CONH [symbol] |
| Ao-CRA3020 | Ao-HsDG-D-N [symbol] -FTDSY [symbol] SRYRKO-N [symbol] -AVK [symbol] KYLAAVL-Ser(Glc)-CONH [symbol] |
| CRA3021 | HsD [symbol] FTDSY₁₀SRYRKO-N [symbol] -AVK [symbol] KYLAAVL-Ser(Glc)-CONH [symbol] |
| Ao-CRA3021 | Ao-HsD [symbol] FTDSY₁₀SRYRKO-N [symbol] -AVK [symbol] KYLAAVL-Ser(Glc)-CONH [symbol] |
| 2L [symbol] 140 | HSDGIFTDSY₁₀SRYRKOLAVK [symbol] KYLAAV [symbol] -Ser(Glc)-Ser(Glc)-CONH [symbol] |

[symbol]

indicates data missing or illegible when filed

### Example 3 In Vivo Stability and BBB Penetration

Initial investigations into the stability of select 2nd series PACAPglycopeptides were carried out in water and in artificial cerebrospinal fluid (aCSF). The compounds remained stable in water, and they exhibited half-life values ranging between 10 and 15 min in aCSF. Selected 2LS-glycosides, namely the glucoside and lactoside, were examined for their stability in rat serum at 37° C. Upon introduction of a glucose residue (2LS72-4) the half-life was extended by approximately 15 min compared to the non-glycosylated compound. The lactose-containing glycopeptide 2LS80Lac exhibited a half-life extending greater than 1 h, suggesting that disaccharides provide a greater degree of protection from enzymatic degradation compared to a monosaccharide. Selected compounds from the 3rd series of PACAP glycopeptides were subjected to in vitro stability studies in rat serum at physiological temperatures as well (Liu et al., 2022) and unpublished results).

The d8 mass shifted analogues of 2LS72-2 and 2LS80Lac (d82LS98-OH and d82LS98Lact, respectively) were investigated for their in vivo BBB transport in rats. More specifically, a technique known as shotgun microdialysis, wherein multiple compounds are injected at one time in a single rat, was utilized to reduce the total number of animals used in the study and to mitigate subtle variations in the injection site (Mabrouk et al., 2012; Hay et al., 2019). Following i.v. injection (15 mg/kg) of d82LS98-OH and d82LS98Lact, aliquots of CSF were removed at specific time points and the concentrations of the peptides were quantified by LC-MS. A dosage of 15 mg/kg was selected due to the high detection limit in the CSF. The concentration of the un-glycosylated compound d82LS98-OH was exceptionally low, suggesting poor penetration of the BBB. However, the CSF concentration of the lactose-containing d82LS98Lact was determined to be much higher, peaking around 400 nM (FIG. 7A). AUC analysis of d82LS98-OH and d82LS98Lact CSF concentrations (normalized to d82LS98-OH) demonstrate the difference in BBB penetration of the two compounds (FIG. 7B). These results strongly indicate that the presence of a carbohydrate motif significantly enhances the penetration of glycopeptides across the BBB compared to their non-glycosylated counterparts. Unlike previous glycopeptides investigated in our lab, native PACAP itself is able to penetrate the BBB to a limited extent. Specifically, PACAP1-38 passes through the BBB via the PTS-6 transporter, while PACAP1-27 is hypothesized to penetrate via a non-saturable diffusion mechanism, albeit at a lower efficiency compared to PACAP1-38, since no such PACAP1-27-selective transporter is known to exist (Banks et al., 1993).

### Example 4 Neuroprotection and Anti-Inflammatory Activity of the Glycopeptides

Following extensive in vitro and in vivo experiments assessing the functional activity and stability and BBB transport of our PACAP glycopeptides, two compounds were chosen for assessment in animal models of TBI and PD. More specifically, 2LS80Mel was chosen for evaluation in a rodent model of TBI, and 2LS98Lac was examined for its neuroprotective potential in the progressive 6-hydroxydopamine (6-OHDA) hemi-parkinsonian rat model.

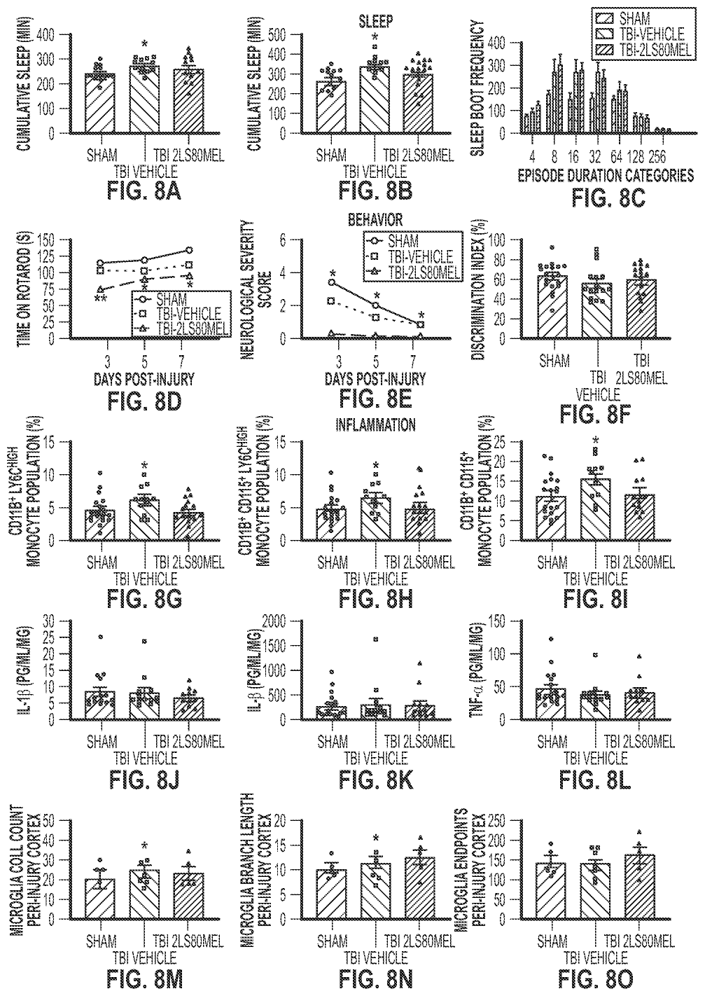

Lead compound 2LS80Mel was evaluated for its neuroprotective potential in vivo utilizing a mouse model of traumatic brain injury (TBI). Mice were pretreated with either a single dose of 2LS80Mel (10 mg/kg i.p.) or sterile saline 30 min prior to being subjected to a midline fluid percussion injury. The mice were then evaluated for sleep-wake behavior (FIGS. 8A-C), motor skills and cognition, (FIGS. 8D-F), the presence of inflammatory markers, (FIGS. 8G-L), and microglial morphology (FIGS. 8M-O).

Rowe and others have previously reported diffuse TBI increases post-traumatic sleep immediately following injury, and attenuation of TBI-induced sleep using pharmacological therapies improved outcomes (Rowe et al., 2014c; Rowe et al., 2018; Rowe et al., 2019). In this study, diffuse TBI led to an increase in cumulative minutes slept in the first 6 h post-injury, and mice administered 2LS80Mel prior to TBI slept comparable minutes to uninjured sham animals (F_{2,46}=3.364, p=0.0433; FIG. 8A). Similarly, TBI led to an increase in sleep in the first dark period, when mice are typically awake, and 2LS80Mel prevented this injury-induced increase in sleep (F_{2,46}=7.710, p=0.0013; FIG. 8B). It has been demonstrated that brain-injured mice sleep more but have shorter, fragmented bouts of sleep, so we analyzed sleep fragmentation as previously published (Rowe et al., 2019; Giordano et al., 2020). The frequency of individual sleep bouts with different episode durations was analyzed and all brain-injured mice had more short bouts (<˜1 min) compared to uninjured shams (F_{2,46}=9.301, p=0.0004; FIG. 8C).

To assess motor function, the Rotarod test was used as previously published by Rowe and others (Rowe et al., 2014b; Rowe et al., 2018). Motor function was tested as the latency to stay on the Rotarod over 7 DPI, with significant effects of both time post-injury (F_{2,110}=8.941, p=0.0003; FIG. 8D) and between treatment groups (F_{2,53}=7.876, p=0.001). Tukey's post hoc analysis indicated that compared to uninjured shams, TBI-vehicle mice had significantly shorter latencies to fall from the rod at 2, 5, and 7 DPI. 2LS80Mel prevented this motor deficit, and TBI-2LS80Mel mice had latencies to fall from the rod that were comparable to uninjured shams at all time points post-injury. Diffuse TBI also resulted in gross neurological dysfunction evaluated by the modified NSS, as we have previously published (Rowe et al., 2014b; Rowe et al., 2018). TBI-vehicle mice had sensorimotor deficits at all time points compared to shams, indicated by a high NNS score. These deficits were prevented by the administration of 2LS80Mel (Fᵣ=6.0, p=0.028; FIG. 8E). There were no TBI-induced cognitive deficits among any group measured by the NOR task (F_{2,53}=1.129, p=0.331; FIG. 8F). Overall, 2LS80Mel successfully attenuated motor skill and cognitive deficits induced by TBI.

Analysis of inflammatory markers and microglial morphology in the different groups of mice further confirmed protective/anti-inflammatory activity of 2LS80Mel. TBI resulted in higher CD11b⁺Ly6C^{high} (F_{2,49}=4.259, p=0.019; FIG. 8G) and CD11b⁺CD115⁺Ly6C^{high} (F_{2,49}=3.250, p=0.047; FIG. 8H) monocyte populations compared to uninjured shams. This TBI-induced increase was not seen in mice treated with 2LS80Mel prior to injury. There were no differences in neutrophil populations among groups (F_{2,49}=0.0159, p=0.984; FIG. 8I). Measurements of pro-inflammatory cytokines revealed no differences in IL-1β (F_{2,41}=0.6789, p=0.513; FIG. 8), IL-6 (F_{2,45}=0.0745, p=0.928; FIG. 8K), or TNF-α (F_{2,45}=0.8546, p=0.432; FIG. 8L) at 2 DPI among groups. There were also no differences in these peripheral cytokines at 14 DPI (data not shown). Microglial ramification was quantified at 15 DPI. Diffuse TBI did not alter microglia cell number (F_{2,14}=0.3795, p=0.691; FIG. 8M), microglia branch length per cell (F_{2,14}=0.7496, p=0.491; FIG. 8N), or microglia endpoints per cell (F_{2,14}=0.4528, p=0.645; FIG. 8O) measured in the peri-injury cortex at 15 DPI. There were no measurable differences in the S1BF or perirhinal cortex at 15 DPI (data not shown).

Overall, 2LS80Mel demonstrated protective effects in a mouse model of diffuse TBI. A single injection of 2LS80Mel resulted in attenuation of common sensorimotor and motor deficits observed in TBI and TBI-induced post-traumatic sleep. Furthermore, TBI-induced increases in peripheral monocyte populations were mitigated by 2LS80Mel. Despite no significant differences between 2LS80Mel-treated and saline-treated groups in levels of select inflammatory cytokines and microglial morphology, 2LS80Mel still demonstrated robust neuroprotection and anti-inflammatory activity in vivo.

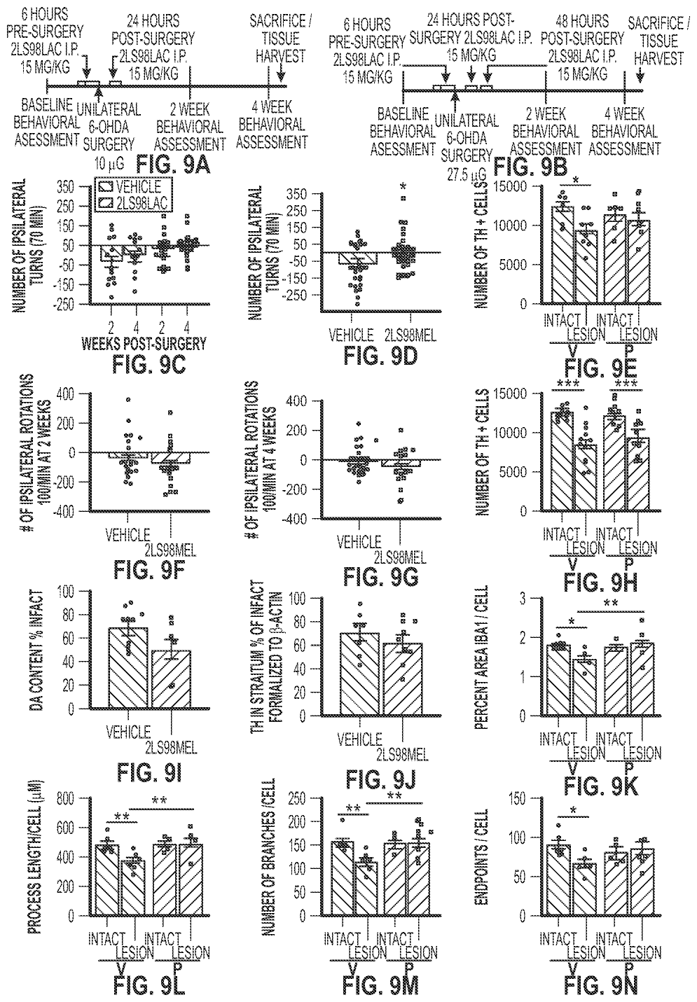

The classical mild progressive 6-OHDA hemi-parkinsonian rat model was used to assess the neuroprotective potential of 2LS98Lac (FIGS. 9A,B). Briefly, the rats were given unilateral lesions by treatment with 6-OHDA. Then, the rats were challenged with a dopaminergic receptor agonist (amphetamine in our case) and assessed for their turning behavior (Kelly et al., 1975; Torres and Dunnet, 2007; DaCunha et al., 2008) The observed rotational behavior is likely due to the denervation of dopaminergic neurons and subsequent hypersensitivity of the dopaminergic receptors in the lesioned area of the brain. In the first experiment, mild lesions (25%) were induced (FIG. 9A) to simulate the early stages of PD, and amphetamine was used to induce contralateral rotations. Rats were treated with either vehicle or 2LS98Lac (15 mg/kg, i.p.) 6 h prior to lesioning and 24 h post-lesion. The total number of cumulative contralateral rotations significantly decreased (t=2.55; p=0.013) in the 2LS98Lac-treated rats compared to vehicle-treated rats (FIG. 9C and FIG. 9D). Following behavioral analysis, the number of tyrosine hydroxylase (TH)-positive neurons in the SNpc of both vehicle-treated and 2LS98Lac-treated animals was determined using immunohistochemical staining and unbiased stereology. While there was a significant reduction of TH-positive cells in the lesioned vs. the intact side in vehicle-treated animals, indicating successful lesioning (F_{3,28}=3.024, p=0.0199), the lack of such a difference in the 2LS98Lac-treated rats indicates that neuronal cell death was attenuated by 2LS98Lac treatment (FIG. 9E). Both behavioral and histological data obtained from these experiments indicate that 2LS98Lac successfully elicited neuroprotection.

In a follow-up study, the neuroprotective potential of 2LS98Lac was also probed in rats with more severe (40%) 6-OHDA-induced lesions and three treatment-injections with 2LS98Lac (FIG. 9B). In this study, we did not see a difference in the amphetamine-induced rotations at either 2 weeks (F₂₁₋₂₂=1.385, p=0.3811; FIG. 9F), or 4 weeks post-lesion (F₂₁₋₂₁=1.070, p=0.3005; FIG. 9G) between vehicle and 2LS98Lac. While in the nigral TH-cell count a mean increase of 850 cells on the lesioned side in the 2LS98Lac group vs. the vehicle group (9,244 vs. 8,391) was seen, this proved to be not significant between the lesioned sides of treatment vs. vehicle (F₃₋₅₀=17.11, p=0.2208; FIG. 9H). There also was no significant difference between treatment and vehicle in striatal TH (F₇₋₇=1.050, p=0.3362; FIG. 9I), as measured with western analysis, or striatal DA content (F₇₋₇=1.748, p=0.0798; FIG. 9J), indicating lack of meaningful protection against the more severe insult.

The microglia morphology was also evaluated in this experiment, to investigate effects on neuroinflammation, and found that 6-OHDA-lesioning did lead to a significant change in the morphologic phenotype that was fully reversed by treatment with 2LS98Lac (FIGS. 9K-N), indicating modulation of neuroinflammation. Specifically, 6-OHDA did lead to reduction of % area IBA-1/cell (F₃₋₂₄=4.463, p=0.0140; FIG. 9K), process length/cell (F₃₋₂₄=5.925, p=0.0057; FIG. 9L), branches/cell (F₃₋₂₄=5.845, p=0.0042; FIG. 9M), and endpoints/cell (F₃₋₂₄=2.943, p=0.0286; FIG. 9N) on the lesioned side. Treatment with 2LS98Lac did lead to an increase of the parameters on the lesioned side back to levels on the intact side. Specifically, there was a significant increase on the lesioned side in the treatment vs. the vehicle group in the % area IBA-1/cell (F₃₋₂₄=4.463, p=0.0087; FIG. 9K), process length/cell (F₃₋₂₄=5.925, p=0.0057; FIG. 9L), branches/cell (F₃₋₂₄=5.845, p=0.0056; FIG. 9M), and a trend toward increase in endpoints/cell (F₃₋₂₄=4.463, p=0.0690; FIG. 9N). Example images for the nigral IHC for TH and for IBA-1, as well as the semi-quantitative western analyses of striatal TH and beta-actin are presented in FIGS. 10A-D.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A glycopeptide comprising a polypeptide and one or more saccharide moieties covalently linked to said polypeptide, said glycopeptide being capable of crossing human blood brain barrier (BBB), said polypeptide having a sequence of:
(SEQ ID NO: 5)
HSDX⁴X⁵FX⁷DSYSRYRKQX¹⁷AVKKYLAAX²⁶X²⁷x²⁸
wherein
X⁴ is glycine; alanine; D-alanine; sarcosine; β-alanine; or diaminovaleric acid;
X⁵ is isoleucine; valine; leucine; L-tert-Leucine; L-nor-Valine; L-Nor-Leucine; glycine;
sarcosine; D- or L-alanine; or D- or L-N-methylalanine;
X⁷ is threonine or alanine;
X¹⁷ is L-Nor-Leucine, L-Leucine; Valine; Nor-Valine; Alanine; or another aliphatic amino acid, or Glycine;
X²⁶ is valine or leucine;
X²⁷ is leucine or serine; and
X²⁸ is absent or serine,
and wherein at least one amino acid residue of said polypeptide is glycosylated.

2. The glycopeptide of claim 1, wherein serine at 2-position is (D)-isomer.

3. The glycopeptide of claim 1, wherein X²⁸ is serine.

4. The glycopeptide of claim 3, wherein X²⁸ is glycosylated with glucose, galactose, melibiose, xylose, lactose, trehalose, or altose.

5. The glycopeptide of claim 1, wherein X¹⁷ is L-Nor-Leucine, or L-Leucine.

6. The glycopeptide of claim 1, wherein X¹⁷ is L-Leucine.

7. The glycopeptide of claim 1, wherein said polypeptide has a sequence selected from the group consisting of:
(SEQ ID NO 6)
HSDGIFTDSYSRYRKQLAVKKYLAAVL-Ser-CONH₂,
and
(SEQ ID NO 7)
HSDGIFTDSYSRYRKQLAVKKYLAAVL-Ser-CONH₂,
wherein Ser-28 and at least one amino acid other than Ser-28 are glycosylated.

8. The glycopeptide of claim 1, wherein said glycopeptide is a pituitary adenylate cyclase-activating polypeptide type I receptor (PAC1) agonist.

9. The glycopeptide of claim 1, wherein said glycopeptide is a VPAC₁ agonist.

10. The glycopeptide of claim 1, wherein said glycopeptide is a selective PAC1 and VPAC1 agonist.

11. The glycopeptide of claim 1, wherein PAC1 binding affinity (Ki) of said glycopeptide is less than about 10 nM.

12. The glycopeptide of claim 1, wherein VPAC1 binding affinity (Ki) of said glycopeptide is less than about 10 nM.

13. The glycopeptide of claim 1, wherein said polypeptide comprises more than one glycosylated amino acid residue.

14. The glycopeptide of claim 1, wherein said saccharide is selected from the group consisting of glucose, maltose, lactose, melibiose, maltotriose, sucrose, trehalose, altose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn).

15. The glycopeptide of claim 1, wherein said glycopeptide is capable of penetrating blood-brain barrier (BBB) and reaching a CSF concentration of at least 50 nM, or 100 nM, or 400 nM 60 minutes after being injected into a subject intravenously at a concentration of 15 mg glycopeptide per kg body weight of said subject.

16. A method for treating a neurodegenerative disease in a subject, said method comprising administering to the subject in need of such a treatment a therapeutically effective amount of a glycopeptide of claim 1.

17. The method of claim 16, wherein said glycopeptide has a higher blood-brain barrier (BBB) penetration compared to said peptide in the absence of said saccharide.

18. The method of claim 16, wherein said glycopeptide is capable of penetrating blood-brain barrier (BBB) and reaching a CSF concentration of at least 50 nM, or 100 nM, or 400 nM 60 minutes after being injected into a subject intravenously or subcutaneously at a concentration of 15 mg glycopeptide per kg body weight of said subject.


SEQUENCE LISTING (106 sequences)

SEQ ID NO: 1 - protein, 38 aa, synthetic construct
HSDGIFTDSYSRYRKQMAVKKYLAAVLGKRYKQRVKNK

SEQ ID NO: 2 - protein, 27 aa, synthetic construct
HSDGIFTDSYSRYRKQMAVKKYLAAVL

SEQ ID NO: 3 - protein, 28 aa, synthetic construct
HSDAVFTDNYTRLRKQMAVKYYLNSILN

SEQ ID NO: 4 - protein, 28 aa, synthetic construct
  VARIANT 4..5: note = X can be any amino acid
  VARIANT 7: note = X can be any amino acid
  VARIANT 17: note = X can be any amino acid
  VARIANT 26..28: note = X can be any amino acid
HSDXXFXDSYSRYRKQXAVKKYLAAXXX

SEQ ID NO: 5 - protein, 28 aa, synthetic construct
  VARIANT 28: note = X can be S or absent
  VARIANT 27: note = L can be replaced by S
  VARIANT 26: note = V can be replaced by L
  VARIANT 17: note = X can be Nle, L-Leucine, V, Nva, A, one of the other aliphatic amino acids or G
  VARIANT 7: note = T can be replaced by A
  MOD_RES 17: note = Nva
  SITE 17: note = Nle
  VARIANT 5: note = I can be replaced by V, L, L-tert-Leucine, Nva, Nle or G
  MOD_RES 5: note = Nva
  SITE 5: note = Nle
  VARIANT 4: note = G can be replaced by A, D-alanine, MeGly, bAla or diaminovaleric acid
  SITE 4: note = D-alanine
  MOD_RES 4: note = MeGly, bAla or diaminovaleric acid
HSDGIFTDSYSRYRKQXAVKKYLAAVLX

SEQ ID NO: 6 - protein, 28 aa, synthetic construct
HSDGIFTDSYSRYRKQLAVKKYLAAVLS

SEQ ID NO: 7 - protein, 28 aa, synthetic construct
HSDGIFTDSYSRYRKQLAVKKYLAAVLS

SEQ ID NO: 8 - protein, 10 aa, synthetic construct
HSDGIFTDSY

SEQ ID NO: 9 - protein, 28 aa, synthetic construct
  CARBOHYD 28: note = Melibiose
HSDGIFTDSYSRYRKQLAVKKYLAAVLS

SEQ ID NO: 10 - protein, 28 aa, synthetic construct
  CARBOHYD 28: note = Glycosylation site
  SITE 17: note = Nle
  SITE 2: note = D-Serine
HSDGIFTDSYSRYRKQXAVKKYLAAVLS

SEQ ID NO: 11 - protein, 27 aa, synthetic construct
  CARBOHYD 27: note = Glycosylation site
  SITE 17: note = Nle
  SITE 2: note = D-Serine
HSDGIFTDSYSRYRKQXAVKKYLAAVS

SEQ ID NO: 12 - protein, 28 aa, synthetic construct
  CARBOHYD 27: note = Glycosylation site
  SITE 17: note = Nle
  SITE 2: note = D-Serine
HSDGIFTDSYSRYRKQXAVKKYLAALSL

SEQ ID NO: 13 - protein, 28 aa, synthetic construct
  CARBOHYD 28: note = Glycosylation site
  SITE 17: note = Nle
  SITE 2: note = D-Serine
HSDGIFADSYSRYRKQXAVKKYLAAVLS

SEQ ID NO: 14 - protein, 27 aa, synthetic construct
  CARBOHYD 27: note = Glycosylation site
  SITE 17: note = Nle
  SITE 2: note = D-Serine
HSDGIFADSYSRYRKQXAVKKYLAAVS

SEQ ID NO: 15 - protein, 28 aa, synthetic construct
  CARBOHYD 27: note = Glycosylation site
  SITE 17: note = Nle
  SITE 2: note = D-Serine
HSDGIFADSYSRYRKQXAVKKYLAAVSL

SEQ ID NO: 16 - protein, 28 aa, synthetic construct
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 28: note = L-Ser(Beta-Melibiose)
HSDGIFTDXYXRYRKQLAVKKYLAAVLX

SEQ ID NO: 17 - protein, 28 aa, synthetic construct
  SITE 2: note = D-Serine
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  SITE 17: note = Nle
  MOD_RES 28: note = L-Ser(Beta-D-Glc)
HSDGIFTDXYXRYRKQXAVKKYLAAVLX

SEQ ID NO: 18 - protein, 28 aa, synthetic construct
  SITE 2: note = D-Serine
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  SITE 17: note = Nle
  MOD_RES 28: note = L-Ser(Beta-D-Glc)
  MOD_RES 1: note = Acetylation
HSDGIFTDXYXRYRKQXAVKKYLAAVLX

SEQ ID NO: 19 - protein, 27 aa, synthetic construct
  SITE 2: note = D-Serine
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  SITE 17: note = Nle
  MOD_RES 27: note = L-Ser(Beta-D-Glc)
HSDGIFTDXYXRYRKQXAVKKYLAAVX

SEQ ID NO: 20 - protein, 28 aa, synthetic construct
  SITE 2: note = D-Serine
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  SITE 17: note = Nle
  MOD_RES 27: note = L-Ser(Beta-D-Glc)
HSDGIFTDXYXRYRKQXAVKKYLAALXL

SEQ ID NO: 21 - protein, 28 aa, synthetic construct
  SITE 2: note = D-Serine
  SITE 17: note = Nle
  MOD_RES 28: note = L-Ser(Beta-D-Glc)
HSDGIFADSYSRYRKQXAVKKYLAAVLX

SEQ ID NO: 22 - protein, 27 aa, synthetic construct
  SITE 2: note = D-Serine
  SITE 17: note = Nle
  MOD_RES 27: note = L-Ser(Beta-D-Glc)
HSDGIFADSYSRYRKQXAVKKYLAAVX

SEQ ID NO: 23 - protein, 28 aa, synthetic construct
  SITE 2: note = D-Serine
  SITE 17: note = Nle
  MOD_RES 27: note = L-Ser(Beta-D-Glc)
HSDGIFADSYSRYRKQXAVKKYLAAVXL

SEQ ID NO: 24 - protein, 28 aa, synthetic construct
  SITE 2: note = D-Serine
  SITE 4: note = D-Alanine
  SITE 17: note = Nle
  MOD_RES 28: note = L-Ser(Beta-D-Glc)
HSDAIFTDSYSRYRKQXAVKKYLAAVLX

SEQ ID NO: 25 - protein, 28 aa, synthetic construct
  SITE 2: note = D-Serine
  MOD_RES 4: note = MeGly
  SITE 17: note = Nle
  MOD_RES 28: note = L-Ser(Beta-D-Glc)
HSDXIFTDSYSRYRKQXAVKKYLAAVLX

SEQ ID NO: 26 - protein, 28 aa, synthetic construct
  SITE 2: note = D-Serine
  MOD_RES 4: note = bAla
  SITE 17: note = Nle
  MOD_RES 28: note = L-Ser(Beta-D-Glc)
HSDXIFTDSYSRYRKQXAVKKYLAAVLX

SEQ ID NO: 27 - protein, 28 aa, synthetic construct
  SITE 2: note = D-Serine
  MOD_RES 4: note = Diaminovaleric Acid
  SITE 17: note = Nle
  MOD_RES 28: note = L-Ser(Beta-D-Glc)
HSDXIFTDSYSRYRKQXAVKKYLAAVLX

SEQ ID NO: 28 - protein, 28 aa, synthetic construct
  SITE 2: note = D-Serine
  SITE 17: note = Nle
  MOD_RES 28: note = L-Ser(Beta-D-Glc)
HSDAIFTDSYSRYRKQXAVKKYLAAVLX

SEQ ID NO: 29 - protein, 28 aa, synthetic construct
  SITE 2: note = D-Serine
  SITE 17: note = Nle
  MOD_RES 28: note = L-Ser(Beta-D-Glc)
HSDBIFTDSYSRYRKQXAVKKYLAAVLX

SEQ ID NO: 30 - protein, 28 aa, synthetic construct
  SITE 2: note = D-Serine
  SITE 17: note = Nle
  MOD_RES 28: note = L-Ser(Beta-D-Glc)
HSDGVFTDSYSRYRKQXAVKKYLAAVLX

SEQ ID NO: 31 - protein, 28 aa, synthetic construct
  SITE 2: note = D-Serine
  SITE 17: note = Nle
  MOD_RES 28: note = L-Ser(Beta-D-Glc)
HSDGLFTDSYSRYRKQXAVKKYLAAVLX

SEQ ID NO: 32 - protein, 28 aa, synthetic construct
  SITE 2: note = D-Serine
  SITE 17: note = Nle
  MOD_RES 28: note = L-Ser(Beta-D-Glc)
HSDGAFTDSYSRYRKQXAVKKYLAAVLX

SEQ ID NO: 33 - protein, 28 aa, synthetic construct
  SITE 2: note = D-Serine
  MOD_RES 5: note = L-tert-Leucine
  SITE 17: note = Nle
  MOD_RES 28: note = L-Ser(Beta-D-Glc)
HSDGXFTDSYSRYRKQXAVKKYLAAVLX

SEQ ID NO: 34 - protein, 28 aa, synthetic construct
  SITE 2: note = D-Serine
  MOD_RES 5: note = Nva
  SITE 17: note = Nle
  MOD_RES 28: note = L-Ser(Beta-D-Glc)
HSDGXFTDSYSRYRKQXAVKKYLAAVLX

SEQ ID NO: 35 - protein, 28 aa, synthetic construct
  SITE 2: note = D-Serine
  SITE 5: note = Nle
  SITE 17: note = Nle
  MOD_RES 28: note = L-Ser(Beta-D-Glc)
HSDGXFTDSYSRYRKQXAVKKYLAAVLX

SEQ ID NO: 36 - protein, 28 aa, synthetic construct
  SITE 2: note = D-Serine
  SITE 17: note = Nle
  MOD_RES 28: note = L-Ser(Beta-D-Glc)
HSDGGFTDSYSRYRKQXAVKKYLAAVLX

SEQ ID NO: 37 - protein, 28 aa, synthetic construct
  SITE 2: note = D-Serine
  MOD_RES 4: note = MeGly
  SITE 17: note = Nle
  MOD_RES 28: note = L-Ser(Beta-D-Glc)
HSDXGFTDSYSRYRKQXAVKKYLAAVLX

SEQ ID NO: 38 - protein, 28 aa, synthetic construct
  SITE 2: note = D-Serine
  MOD_RES 5: note = MeGly
  SITE 17: note = Nle
  MOD_RES 28: note = L-Ser(Beta-D-Glc)
HSDGXFTDSYSRYRKQXAVKKYLAAVLX

SEQ ID NO: 39 - protein, 28 aa, synthetic construct
  SITE 2: note = D-Serine
  MOD_RES 4..5: note = MeGly
  SITE 17: note = Nle
  MOD_RES 28: note = L-Ser(Beta-D-Glc)
HSDXXFTDSYSRYRKQXAVKKYLAAVLX

SEQ ID NO: 40 - protein, 28 aa, synthetic construct
  MOD_RES 4: note = MeGly
  SITE 2: note = D-Serine
  SITE 17: note = Nle
  MOD_RES 28: note = L-Ser(Beta-D-Glc)
HSDXAFTDSYSRYRKQXAVKKYLAAVLX

SEQ ID NO: 41 - protein, 28 aa, synthetic construct
  SITE 2: note = D-Serine
  MOD_RES 5: note = MeGly
  SITE 17: note = Nle
  MOD_RES 28: note = L-Ser(Beta-D-Glc)
HSDAXFTDSYSRYRKQXAVKKYLAAVLX

SEQ ID NO: 42 - protein, 28 aa, synthetic construct
  SITE 2: note = D-Serine
  MOD_RES 4: note = MeGly
  SITE 5: note = D-Alanine
  SITE 17: note = Nle
  MOD_RES 28: note = L-Ser(Beta-D-Glc)
HSDXAFTDSYSRYRKQXAVKKYLAAVLX

SEQ ID NO: 43 - protein, 28 aa, synthetic construct
  SITE 2: note = D-Serine
  SITE 4: note = D-Alanine
  MOD_RES 5: note = MeGly
  SITE 17: note = Nle
  MOD_RES 28: note = L-Ser(Beta-D-Glc)
HSDAXFTDSYSRYRKQXAVKKYLAAVLX

SEQ ID NO: 44 - protein, 28 aa, synthetic construct
  SITE 2: note = D-Serine
  MOD_RES 5: note = L-N-Methylalanine
  SITE 17: note = Nle
  MOD_RES 28: note = L-Ser(Beta-D-Glc)
HSDAXFTDSYSRYRKQXAVKKYLAAVLX

SEQ ID NO: 45 - protein, 28 aa, synthetic construct
  SITE 2: note = D-Serine
  SITE 4: note = D-Alanine
  MOD_RES 5: note = L-N-Methylalanine
  SITE 17: note = Nle
  MOD_RES 28: note = L-Ser(Beta-D-Glc)
HSDAXFTDSYSRYRKQXAVKKYLAAVLX

SEQ ID NO: 46 - protein, 28 aa, synthetic construct
  SITE 2: note = D-Serine
  MOD_RES 5: note = D-N-Methylalanine
  SITE 17: note = Nle
  MOD_RES 28: note = L-Ser(Beta-D-Glc)
HSDAXFTDSYSRYRKQXAVKKYLAAVLX

SEQ ID NO: 47 - protein, 28 aa, synthetic construct
  SITE 2: note = D-Serine
  SITE 4: note = D-Alanine
  MOD_RES 5: note = D-N-Methylalanine
  SITE 17: note = Nle
  MOD_RES 28: note = L-Ser(Beta-D-Glc)
  SITE 5: note = D-N-Methylalanine
HSDAXFTDSYSRYRKQXAVKKYLAAVLX

SEQ ID NO: 48 - protein, 28 aa, synthetic construct
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 28: note = L-Ser(Beta-Melibiose)
HSDGIFTDXYXRYRKQMAVKKYLAAVLX

SEQ ID NO: 49 - protein, 28 aa, synthetic construct
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 28: note = each is optionally and independent glycosylated with glucose, galactose, melibiose, xylose, lactose, trehalose, altose or other monosaccharide or a disaccharide.
HSDGIFTDXYXRYRKQMAVKKYLAAVLX

SEQ ID NO: 50 - protein, 28 aa, synthetic construct
  MOD_RES 2: note = each is optionally and independent glycosylated with glucose, galactose, melibiose, xylose, lactose, trehalose, altose or other monosaccharide or a disaccharide.
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 28: note = each is optionally and independent glycosylated with glucose, galactose, melibiose, xylose, lactose, trehalose, altose or other monosaccharide or a disaccharide.
  VARIANT 17: note = M can be replaced by Nle
  SITE 17: note = Nle
HXDGIFTDXYXRYRKQMAVKKYLAAVLX

SEQ ID NO: 51 - protein, 28 aa, synthetic construct
  MOD_RES 2: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 28: note = each is optionally and independent glycosylated with glucose, galactose, melibiose, xylose, lactose, trehalose, altose or other monosaccharide or a disaccharide.
  SITE 2: note = D-Serine
  VARIANT 9: note = D- or L- isomer of serine
  SITE 9: note = D-isomer of serine
HXDGIFTDXYXRYRKQMAVKKYLAAVLX

SEQ ID NO: 52 - protein, 28 aa, synthetic construct
  MOD_RES 2: note = each is optionally and independent glycosylated with glucose, galactose, melibiose, xylose, lactose, trehalose, altose or other monosaccharide or a disaccharide.
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  VARIANT 28: note = D- or L- isomer of serine
  SITE 28: note = D-isomer of serine
HXDGIFTDXYXRYRKQMAVKKYLAAVLX

SEQ ID NO: 53 - protein, 27 aa, synthetic construct
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 27: note = L-Ser(Beta-Melibiose)
HSDGIFTDXYXRYRKQMAVKKYLAAVX

SEQ ID NO: 54 - protein, 27 aa, synthetic construct
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 27: note = each is optionally and independent glycosylated with glucose, galactose, melibiose, xylose, lactose, trehalose, altose or other monosaccharide or a disaccharide.
HSDGIFTDXYXRYRKQMAVKKYLAAVX

SEQ ID NO: 55 - protein, 27 aa, synthetic construct
  MOD_RES 2: note = each is optionally and independent glycosylated with glucose, galactose, melibiose, xylose, lactose, trehalose, altose or other monosaccharide or a disaccharide.
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 27: note = each is optionally and independent glycosylated with glucose, galactose, melibiose, xylose, lactose, trehalose, altose or other monosaccharide or a disaccharide.
HXDGIFTDXYXRYRKQMAVKKYLAAVX

SEQ ID NO: 56 - protein, 27 aa, synthetic construct
  MOD_RES 2: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  MOD_RES 27: note = each is optionally and independent glycosylated with glucose, galactose, melibiose, xylose, lactose, trehalose, altose or other monosaccharide or a disaccharide.
  SITE 2: note = D-Serine
  VARIANT 9: note = D- or L- isomer of serine
  SITE 9: note = D-isomer of serine
  MOD_RES 9: note = each is optionally and independent glycosylated with glucose, galactose, melibiose, xylose, lactose, trehalose, altose or other monosaccharide or a disaccharide.
HXDGIFTDXYXRYRKQMAVKKYLAAVX

SEQ ID NO: 57 - protein, 27 aa, synthetic construct
  MOD_RES 2: note = each is optionally and independent glycosylated with glucose, galactose, melibiose, xylose, lactose, trehalose, altose or other monosaccharide or a disaccharide.
  MOD_RES 9: note = each is optionally and independent glycosylated with glucose, galactose, melibiose, xylose, lactose, trehalose, altose or other monosaccharide or a disaccharide.
  MOD_RES 11: note = each is optionally and independent glycosylated with glucose, galactose, melibiose, xylose, lactose, trehalose, altose or other monosaccharide or a disaccharide.
  VARIANT 27: note = D- or L- isomer of serine
  SITE 27: note = D-isomer of serine
  MOD_RES 27: note = each is optionally and independent glycosylated with glucose, galactose, melibiose, xylose, lactose, trehalose, altose or other monosaccharide or a disaccharide.
HXDGIFTDXYXRYRKQMAVKKYLAAVX

SEQ ID NO: 58 - protein, 13 aa, synthetic construct
PPPPPPPPPPPPP

SEQ ID NO: 59 - protein, 30 aa, synthetic construct
GGGGGGGGGGGGGGGGGGGGGGGGGGGGGG

SEQ ID NO: 60 - protein, 4 aa, synthetic construct
FFFF

SEQ ID NO: 61 - protein, 10 aa, synthetic construct
PPPPPPPPPP

SEQ ID NO: 62 - protein, 11 aa, synthetic construct
KKKKKKKKKKK

SEQ ID NO: 63 - protein, 14 aa, synthetic construct
WWWWWWWWWWWWWW

SEQ ID NO: 64 - protein, 17 aa, synthetic construct
QQQQQQQQQQQQQQQQQ

SEQ ID NO: 65 - protein, 27 aa, synthetic construct
HSDGIFTDSYSRYRKQMAVKKYLAAVL

SEQ ID NO: 66 - protein, 27 aa, synthetic construct
HSDGIFTDSYSRYRKQLAVKKYLAAVL

SEQ ID NO: 67 - protein, 27 aa, synthetic construct
  SITE 2: note = D-Serine
HSDGIFTDSYSRYRKQLAVKKYLAAVL

SEQ ID NO: 68 - protein, 28 aa, synthetic construct
  CARBOHYD 28: note = Glycosylation site
HSDGIFTDSYSRYRKQLAVKKYLAAVLS

SEQ ID NO: 69 - protein, 28 aa, synthetic construct
  CARBOHYD 28: note = Lactose
HSDGIFTDSYSRYRKQLAVKKYLAAVLS

SEQ ID NO: 70 - protein, 28 aa, synthetic construct
  CARBOHYD 28: note = Cellobiose
HSDGIFTDSYSRYRKQLAVKKYLAAVLS

SEQ ID NO: 71 - protein, 28 aa, synthetic construct
  CARBOHYD 28: note = Melibiose
HSDGIFTDSYSRYRKQLAVKKYLAAVLS

SEQ ID NO: 72 - protein, 28 aa, synthetic construct
  SITE 2: note = D-Serine
  MOD_RES 17: note = Nva
  CARBOHYD 28: note = Glycosylation site
HSDGIFTDSYSRYRKQXAVKKYLAAVLS

SEQ ID NO: 73 - protein, 27 aa, synthetic construct
  SITE 2: note = D-Serine
  MOD_RES 16: note = Nva
  CARBOHYD 27: note = Glycosylation site
HSDGIFTDSYSRYRKQXAVKKYLAAVS

SEQ ID NO: 74 - protein, 27 aa, synthetic construct
  SITE 2: note = D-Serine
  MOD_RES 17: note = Nva
  CARBOHYD 26: note = Glycosylation site
HSDGIFTDSYSRYRKQXAVKKYLAASL

SEQ ID NO: 75 - protein, 28 aa, synthetic construct
  SITE 2: note = D-Serine
  MOD_RES 17: note = Nva
  CARBOHYD 28: note = Glycosylation site
HSDGIFADSYSRYRKQXAVKKYLAAVLS

SEQ ID NO: 76 - protein, 27 aa, synthetic construct
  SITE 2: note = D-Serine
  MOD_RES 17: note = Nva
  CARBOHYD 27: note = Glycosylation site
HSDGIFADSYSRYRKQXAVKKYLAAVS

SEQ ID NO: 77 - protein, 27 aa, synthetic construct
  SITE 2: note = D-Serine
  MOD_RES 17: note = Nva
  CARBOHYD 26: note = Glycosylation site
HSDGIFADSYSRYRKQXAVKKYLAASL

SEQ ID NO: 78 - protein, 28 aa, synthetic construct
  CARBOHYD 28: note = Melibiose
HSDGIFTDSYSRYRKQLAVKKYLAAVLS

SEQ ID NO: 79 - protein, 28 aa, synthetic construct
  CARBOHYD 28: note = Cellobiose
HSDGIFTDSYSRYRKQLAVKKYLAAVLS

SEQ ID NO: 80 - protein, 28 aa, synthetic construct
  CARBOHYD 28: note = Lactose
HSDGIFTDSYSRYRKQLAVKKYLAAVLS

SEQ ID NO: 81 - protein, 28 aa, synthetic construct
  SITE 2: note = D-Serine
  MOD_RES 17: note = Nva
  CARBOHYD 28: note = Glycosylation site
  MOD_RES 4: note = GABA
HSDXIFTDSYSRYRKQXAVKKYLAAVLS

SEQ ID NO: 82 - protein, 28 aa, synthetic construct
  SITE 2: note = D-Serine
  MOD_RES 4: note = MeGly
  MOD_RES 17: note = Nva
  CARBOHYD 28: note = Glycosylation site
HSDXIFTDSYSRYRKQXAVKKYLAAVLS

SEQ ID NO: 83 - protein, 28 aa, synthetic construct
  SITE 2: note = D-Serine
  MOD_RES 4: note = bAla
  MOD_RES 17: note = Nva
  CARBOHYD 28: note = Glycosylation site
HSDXIFTDSYSRYRKQXAVKKYLAAVLS

SEQ ID NO: 84 - protein, 28 aa, synthetic construct
  SITE 2: note = D-Serine
  MOD_RES 4: note = Diaminovaleric Acid
  MOD_RES 17: note = Nva
  CARBOHYD 28: note = Glycosylation site
HSDXIFTDSYSRYRKQXAVKKYLAAVLS

SEQ ID NO: 85 - protein, 28 aa, synthetic construct
  SITE 2: note = D-Serine
  MOD_RES 17: note = Nva
  CARBOHYD 28: note = Glycosylation site
HSDAIFTDSYSRYRKQXAVKKYLAAVLS

SEQ ID NO: 86 - protein, 28 aa, synthetic construct
  SITE 2: note = D-Serine
  MOD_RES 4: note = Aib
  MOD_RES 17: note = Nva
  CARBOHYD 28: note = Glycosylation site
HSDXIFTDSYSRYRKQXAVKKYLAAVLS

SEQ ID NO: 87 - protein, 28 aa, synthetic construct
  SITE 2: note = D-Serine
  MOD_RES 17: note = Nva
  CARBOHYD 28: note = Glycosylation site
HSDGVFTDSYSRYRKQXAVKKYLAAVLS

SEQ ID NO: 88 - protein, 28 aa, synthetic construct
  SITE 2: note = D-Serine
  MOD_RES 17: note = Nva
  CARBOHYD 28: note = Glycosylation site
HSDGAFTDSYSRYRKQXAVKKYLAAVLS

SEQ ID NO: 89 - protein, 28 aa, synthetic construct
  SITE 2: note = D-Serine
  MOD_RES 17: note = Nva
  CARBOHYD 28: note = Glycosylation site
HSDGXFTDSYSRYRKQXAVKKYLAAVLS

SEQ ID NO: 90 - protein, 28 aa, synthetic construct
  SITE 2: note = D-Serine
  MOD_RES 17: note = Nva
  CARBOHYD 28: note = Glycosylation site
  MOD_RES 5: note = Tle
HSDGXFTDSYSRYRKQXAVKKYLAAVLS

SEQ ID NO: 91 - protein, 28 aa, synthetic construct
  SITE 2: note = D-Serine
  MOD_RES 5: note = Nva
  MOD_RES 17: note = Nva
  CARBOHYD 28: note = Glycosylation site
HSDGXFTDSYSRYRKQXAVKKYLAAVLS

SEQ ID NO: 92 - protein, 28 aa, synthetic construct
  SITE 2: note = D-Serine
  SITE 5: note = Nle
  MOD_RES 17: note = Nva
  CARBOHYD 28: note = Glycosylation site
HSDGXFTDSYSRYRKQXAVKKYLAAVLS

SEQ ID NO: 93 - protein, 28 aa, synthetic construct
  CARBOHYD 28: note = Lactose
  MOD_RES 28: note = L-Ser(Beta-Melibiose)
HSDGIFTDSYSRYRKQLAVKKYLAAVLS

SEQ ID NO: 94 - protein, 28 aa, synthetic construct
  MOD_RES 1: note = Acetylation
  MOD_RES 28: note = L-Ser(Beta-Melibiose)
  CARBOHYD 28: note = Lactose
HSDGIFTDSYSRYRKQLAVKKYLAAVLS

SEQ ID NO: 95 - protein, 28 aa, synthetic construct
  MOD_RES 1: note = Acetylation
  SITE 2: note = D-Serine
  MOD_RES 17: note = Nva
  CARBOHYD 28: note = Glycosylation site
HSDGIFTDSYSRYRKQXAVKKYLAAVLS

SEQ ID NO: 96 - protein, 28 aa, synthetic construct
  SITE 2: note = D-Serine
  MOD_RES 17: note = Nva
  CARBOHYD 28: note = Lactose
HSDGIFTDSYSRYRKQXAVKKYLAAVLS

SEQ ID NO: 97 - protein, 28 aa, synthetic construct
  MOD_RES 1: note = Acetylation
  SITE 2: note = D-Serine
  MOD_RES 17: note = Nva
  CARBOHYD 28: note = Lactose
HSDGIFTDSYSRYRKQXAVKKYLAAVLS

SEQ ID NO: 98 - protein, 28 aa, synthetic construct
  MOD_RES 4: note = MeGly
  SITE 5: note = Nle
  MOD_RES 17: note = Nva
  CARBOHYD 28: note = Glycosylation site
  SITE 2: note = D-Serine
HSDXXFTDSYSRYRKQXAVKKYLAAVLS

SEQ ID NO: 99 - protein, 28 aa, synthetic construct
  MOD_RES 1: note = Acetylation
  SITE 2: note = D-Serine
  MOD_RES 4: note = MeGly
  SITE 5: note = Nle
  MOD_RES 17: note = Nva
  CARBOHYD 28: note = Glycosylation site
HSDXXFTDSYSRYRKQXAVKKYLAAVLS

SEQ ID NO: 100 - protein, 28 aa, synthetic construct
  SITE 2: note = D-Serine
  SITE 5: note = D-Nle
  MOD_RES 17: note = Nva
  CARBOHYD 28: note = Glycosylation site
  SITE 5: note = Nle
HSDGXFTDSYSRYRKQXAVKKYLAAVLS

SEQ ID NO: 101 - protein, 28 aa, synthetic construct
  MOD_RES 1: note = Acetylation
  SITE 2: note = D-Serine
  SITE 5: note = Nle
  MOD_RES 17: note = Nva
  CARBOHYD 28: note = Glycosylation site
  SITE 5: note = D-Nle
HSDGXFTDSYSRYRKQXAVKKYLAAVLS

SEQ ID NO: 102 - protein, 28 aa, synthetic construct
  SITE 2: note = D-Serine
  MOD_RES 5: note = Nva
  MOD_RES 17: note = Nva
  CARBOHYD 28: note = Glycosylation site
  SITE 5: note = D-Nva
HSDGXFTDSYSRYRKQXAVKKYLAAVLS

SEQ ID NO: 103 - protein, 28 aa, synthetic construct
  MOD_RES 1: note = Acetylation
  SITE 2: note = D-Serine
  MOD_RES 5: note = Nva
  MOD_RES 17: note = Nva
  CARBOHYD 28: note = Glycosylation site
  SITE 5: note = D-Nva
HSDGXFTDSYSRYRKQXAVKKYLAAVLS

SEQ ID NO: 104 - protein, 28 aa, synthetic construct
  SITE 2: note = D-Serine
  SITE 4: note = D-Alanine
  SITE 5: note = D-Leucine
  MOD_RES 17: note = Nva
  CARBOHYD 28: note = Glycosylation site
HSDALFTDSYSRYRKQXAVKKYLAAVLS

SEQ ID NO: 105 - protein, 28 aa, synthetic construct
  MOD_RES 1: note = Acetylation
  SITE 2: note = D-Serine
  SITE 4: note = D-Alanine
  SITE 5: note = D-Leucine
  MOD_RES 17: note = Nva
  CARBOHYD 28: note = Glycosylation site
HSDALFTDSYSRYRKQXAVKKYLAAVLS

SEQ ID NO: 106 - protein, 29 aa, synthetic construct
  CARBOHYD 28..29: note = Glycosylation site
HSDGIFTDSYSRYRKQLAVKKYLAAVLSS